(19) United States
(12) Patent Application Publication
(10) Pub. No.: US 2023/0285331 A1
(43) Pub. Date: Sep. 14, 2023

(54) TYROSINE AND RESVERATROL DERIVATIVES AS NOVEL MODULATORS OF CELLULAR SERINE-ADP-RIBOSYLATION

(71) Applicant: UNIVERSITY OF SOUTH CAROLINA, Columbia, SC (US)

(72) Inventor: SAJISH MATHEW, LEXINGTON, SC (US)

(21) Appl. No.: 17/993,027

(22) Filed: Nov. 23, 2022

Related U.S. Application Data
(60) Provisional application No. 63/349,673, filed on Jun. 7, 2022.
(60) Provisional application No. 63/302,349, filed on Jan. 24, 2022.

(51) Int. Cl. A61K 31/164; A61K 31/198; A61K 31/405; A61K 31/4164; A61P 25/28

(52) U.S. Cl. CPC A61K 31/164; A61K 31/198; A61K 31/405; A61K 31/4164; A61P 25/28

(57) ABSTRACT

Methods and compositions are described herein for modulating protein synthesis, especially monosome translation and DNA damage response, especially short-patch base excision repair (SP-BER), NHEJ and HR for treating a disorder. Compositions include a therapeutically effective amount of a tyrosine or a resveratrol derivative.

### CROSS REFERENCE TO RELATED APPLICATIONS

This application claims filing benefit of U.S. Provisional Application Ser. No. 63/302,349, having a filing date of Jan. 24, 2022, and U.S. Provisional Application Ser. No. 63/349,673, having a filing date of Jun. 7, 2022, the entire contents of which are incorporated herein by reference.

### FEDERAL RESEARCH STATEMENT

This invention was made with government support under P20 GM109091 awarded by the National Institute of Health. The government has certain rights in the invention.

### BACKGROUND

Aminoacyl-tRNA synthetases activate amino acids for protein synthesis. For example, tyrosyl-tRNA (TyrRS) activates tyrosine for protein synthesis. In addition to L-tyrosine (L-Tyr) being utilized for protein synthesis, L-Tyr also acts as a substrate for tyrosine hydroxylase that catalyzes the rate-limiting step in the synthesis of dopamine. Tyrosine is circadian regulated, with the highest serum levels in the morning and with the lowest at midnight (sleep time). Intriguingly, brain protein synthesis, memory formation, and neuronal DNA repair are activated during sleep when tyrosine levels are decreased during the nadir/trough of circadian rhythm. Tyrosine levels are also modulated by the circadian activities of tyrosine hydroxylase, tyrosine aminotransferases (TAT), and gut microbiota. Genetic mutations that increase the levels of tyrosine (tyrosinemia) or its precursor L-phenylalanine (Phe, phenylketonuria [PKU]) cause multiple health problems, including cognitive deficits in children. Moreover, tyrosine and/or phenylalanine exacerbate cognitive decline in the elderly and in Alzheimer’s disease (AD) patients, in addition to shortening lifespan in tyrosinemia patients. Although protein synthesis is required for long-term memory formation, and brain-derived neurotrophic factor (BDNF) stimulates the de novo synthesis of TyrRS in primary cortical neurons, recent brain proteomic analysis showed that TyrRS is decreased in the affected brain regions of AD patients through an unknown mechanism.

Recent metabolic analyses showed that calorie restriction (CR) decreases serum tyrosine levels, whether tyrosine has any causal effects in aging and age-associated disorders and their reversal during CR is not yet known. Calorie restriction promotes genomic stability through the induction of base excision repair (BER) and reversal of its age-related decline along with extension of lifespan and protection against age-associated neurocognitive and metabolic disorders, including cardiovascular diseases (CVD). Most importantly, the natural molecule resveratrol (RSV) was shown to evoke CR-like health benefits in humans, suggesting that RSV may act as a potential ‘CR mimetic’. Intriguingly, clinical studies using the trans-isomer of RSV (trans-RSV) brought out conflicting outcomes in which lower doses of trans-RSV produced encouraging results, but higher doses exacerbated the diseases. For example, low-dose trans-RSV showed CR-like benefits in obese males, cognitive benefits in AD patients, and in postmenopausal women, in addition to protection against heart failure. However, higher doses of trans-RSV resulted in brain volume loss in AD patients, worsened memory performance in schizophrenia, and increased CVD risk. Despite decades of research, the molecular basis of these controversial effects of trans-RSV (low dose CR-like beneficial effects versus high-dose detrimental effects) remains unknown.

Increased tyrosine and phenylalanine levels decrease TyrRS and cause neuronal oxidative DNA damage by simultaneously inhibiting protein synthesis and DNA repair. Furthermore, cis- and trans-RSV have opposite effects on TyrRS levels, protein synthesis, DNA repair, and survival of rat cortical neurons. cis-RSV protects the neurons against tyrosine/phenylalanine and other neurotoxic agents-induced depletion of TyrRS and DNA damage.

In view of the above, a need exists for improved compositions and methods of modulating metabolic pathways and treating neurocognitive and metabolic disorders.

### SUMMARY

In general, the present disclosure is directed to methods and compositions for use in modulating DNA damage response (DDR) signaling pathways. Further, disclosed herein are methods for modulating and/or treating neurocognitive, sleep, and/or metabolic disorders and various cancerous growth of tissues/cells. The composition and method include administering to a subject in need thereof a therapeutically effective amount of a pharmaceutical composition comprising a compound having Formula (I):

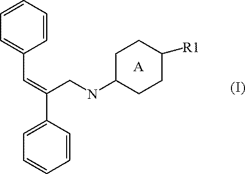

or a pharmaceutically acceptable salt and their isomers thereof, wherein:
ring A is a substituted or unsubstituted carbocyclyl, substituted or unsubstituted heterocyclyl, substituted or unsubstituted aryl, substituted or unsubstituted heteroaryl; and

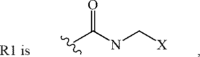

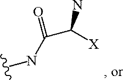

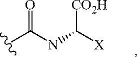

wherein X is an amino acid side chain.

Also, compositions disclosed herein can include a tyrosine derivative compound of formula V:

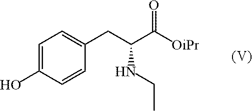 [SMILES: CCN[C@H](Cc1ccc(O)cc1)C(=O)OC(C)C]

or a pharmaceutically acceptable salt and their isomers thereof.

Further, compositions disclosed herein can include a tyrosine derivative compound of formula VI:

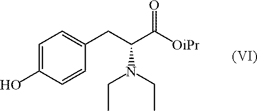 [SMILES: CCN(CC)[C@H](Cc1ccc(O)cc1)C(=O)OC(C)C]

or a pharmaceutically acceptable salt and their isomers thereof.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1A presents spectral images (scale bar, 20 µm) and quantitative immunofluorescence analysis of TyrRS in the nucleus, soma, and neurite of rat cortical neurons (DIV9) using anti-TyrRS antibody after treatment with Tyr (250 µM) for 4 hr.

FIG. 1B presents spectral images (scale bar, 20 µm) and quantitative immunofluorescence analysis of TyrRS in the nucleus, soma, and neurite of rat cortical neurons (DIV9) using anti-TyrRS antibody after treatment with low Tyr medium for 2 hr.

FIG. 1C presents total TyrRS immunoblots after treatment with low Tyr medium in rat cortical neurons (DIV9) using anti-TyrRS antibody.

FIG. 1D presents primary cortical neurons treated with Tyr (100-300 µM) for 4 hr, and the levels of PheRSβ and PheRSα detected by Western Blot analysis using their specific antibodies.

FIG. 1E presents representative images (scale bar, 20 µm) for cortical neurons following siRNA TyrRS treatment for 72 hr (MAP2- neurite marker, and DAPI -nuclear marker).

FIG. 1F presents immunoblots and quantification for TyrRS and PheRSα/β using anti-TyrRS and PheRSα/β antibodies in the hippocampal region of AD patients (n=7) with age and sex-matched controls (n=7).

FIG. 2A presents primary cortical neurons were treated with Tyr (200 µM) for up to 24 hr, and TyrRS was detected by Western Blot analysis using anti-TyrRS antibody.

FIG. 2B presents representative immunoblots for TyrRS and PheRSα/β after treatment with dopamine (100-200 µM) for up to 30 min in rat cortical neurons (DIV9).

FIG. 2C presents representative immunoblots for TyrRS and PheRSβ after treatment with either dopamine (100 µM) alone or in combination with rapamycin (100 nM) in rat cortical neurons (DIV9).

FIG. 2D presents primary cortical neurons treated with Tyr (100-500 µM) for 8 hr and p-eEF2 was detected by Western Blot analysis using anti-p-eEF2 antibody.

FIG. 3A presents immunoblots and spectral images (scale bar, 20 µm) and quantification for TyrRS after treatment with cis-and trans-RSV (10 and 50 µM) for up to 16 hr in rat cortical neurons (DIV9).

FIG. 3B presents spectral images (scale bar, 20 µm) the protein level of neuronal TyrRS in rat cortical neurons (DIV10) following treatment with either cis-RSV or trans-RSVfor 16 hr (MAP2 - neurite marker; DAPI - nuclear marker; TyrRS).

FIG. 3C presents immunoblots showing the protein levels of TyrRS after treatment with trans-RSV (50 µM) alone or in combination with different doses of cis-RSV for 16 hr in rat cortical neurons (DIV9).

FIG. 3D presents primary cortical neurons treated with Tyr (200 µM) alone or in combination with cis-RSV for 8 hr, and TyrRS was detected by Western Blot using anti-TyrRS antibody.

FIG. 3E presents immunoblots and quantification for PheRSα/β after treatment with cis-and trans-RSV (5-50 µM) for up to 16 hr in rat cortical neurons (DIV9).

FIG. 3F presents primary cortical neurons treated with cis-RSV (50 µM) for 30 min either alone or in combination with CHX (100 µM).

FIG. 3G presents primary cortical neurons treated with cis- and trans-RSV (5-50 µM) for 8 hr and p-eEF2 was detected by Western Blot analysis using anti-p-eEF2 antibody.

FIG. 3H presents primary cortical neurons treated with cis- and trans-RSV (5-50 µM) for 1 hr and quantifying the levels of puromycin using anti-puromycin antibody.

FIG. 3I presents primary cortical neurons treated with cis- and trans-RSV (50 µM) or Tyr (200 µM) either alone or in combination with cis-RSV. Immunoprecipitated eEF2 was probed for its interaction with PP2A and TyrRS using their specific antibodies.

FIG. 4A presents immunostaining images (scale bar, 10 µm) for DNA damage marker, pSer139-H2AX foci (y-H2AX; DAPI - nuclear marker) in cortical neurons (DIV10) after treatment with cis- and trans-RSV (50 µM) alone or in combination with Tyr (250 µM) for 24 hr.

FIG. 4B presents quantification of 8-oxo-2′-dG levels using immunofluorescence (IF) in rat primary cortical neurons (DIV9/10) after treatment with Tyr (500 µM) either alone or in combination with cis or trans-RSV (50 µM) for 16 hr.

FIG. 4C presents primary cortical neurons treated with Tyr (100-300 µM) for 8 hr, and OGG1 was detected by Western Blot analysis using anti-OGG1 antibody.

FIG. 4D presents rat cortical neurons (DIV9) treated with either D-Tyr alone or in combination with cis or trans-RSV (50 µM) for 48 hr, and viability was assessed using MTT assay.

FIG. 4E presents representative images (scale bar, 20 µm) for cortical neurons following D-Tyr or cis-RSV and trans-RSV (50 µM) for 24 hr treatment (MAP2 -neurite marker, and DAPI - nuclear marker).

FIG. 4F presents rat cortical neurons (DIV9-10) treated with cis-RSV (50 µM), trans-RSV (50 µM), L-Tyr (1 mM) either alone or in combination for 1 hr.

FIG. 5A presents immunoblots showing the protein levels of HPF1 in rat cortical neurons (DIV9-10) after treatment with Tyr (≤ 300 µM) for up to 8 hr.

FIG. 5B presents representative immunoblots and quantification of histone H3-Ser10- ADP-ribosylation levels in cortical neurons (DIV9) using anti-H3-Ser10-ADPR antibody after treatment with D-tyrosine (2.5 and 5 mM).

FIG. 5C presents representative immunoblots and quantification for HPF1 using anti-HPF1 antibodies in cortical neurons after treatment with cis-and trans-RSV (25-50 µM).

FIG. 5D presents representative immunoblots and quantification for histone H3-Serine-ADPR using anti-H3-Ser10-ADPR antibodies in cortical neurons after treatment with cis-and trans-RSV (25-50 µM).

FIG. 5E presents representative immunoblots and quantification for HPF1 and histone H3-Serine-ADPR using anti-HPF1 and anti-H3-Serine-ADPR antibodies respectively in cortical neurons after treatment with BDNF (50 nM).

FIG. 5F presents representative immunoblots and quantification for HPF1 and histone H3-Serine-ADPR using anti-HPF1 and anti-H3-Serine-ADPR antibodies respectively in cortical neurons after treatment with ISRIB (5 and 10 nM).

FIG. 5G presents representative immunoblots and quantification of histone H3-Ser10-ADPR levels in cortical neurons (DIV9) using anti-H3-Ser10-ADPR antibody after treatment with DA (250 µM) for 5-10 mins.

FIG. 5H presents representative immunoblots and quantification for HPF1 and histone H3-Ser10-ADP-ribosylation (Serine-ADPR) using anti-HPF1 and anti-H3-Ser10-ADPR antibodies respectively in the hippocampal region of AD patients (n=5) with age and sex-matched controls (n=5).

FIG. 6 presents derivatives of tyrosine and resveratrol.

FIG. 7 presents average serum tyrosine levels in females compared to males.

FIG. 8A presents primary cortical neurons treated with Tyr (100-300 µM) for 4 hr, and the levels of PheRSβ and PheRSα detected by Western Blot analysis using their specific antibodies.

FIG. 8B presents primary cortical neurons treated with phenylalanine (100-300 µM) for 8 hr, and the levels of TyrRS, and PheRSα/β were detected by Western Blot analysis using their specific antibodies.

FIG. 8C presents representative immunoblots showing TyrRS, PheRSα/β levels after treatment with increasing concentrations of L-DOPA (100-300 µM) for 8 hr using their specific antibodies.

FIG. 8D presents representative immunoblots showing TyrRS, PheRSβ levels after treatment with increasing concentrations of 6-OHDA (100 and 200 µM) for 8 hr using their specific antibodies.

FIG. 8E presents rat cortical neurons (DIV7) transfected with siRNA against TyrRS or control siRNA (75 nM) for 72 hr, and the levels of TyrRS were quantified using anti-TyrRS antibody.

FIG. 8F presents primary cortical neurons treated with MG132 (100 µM) for up to 2 hr and the changes in the protein levels of TyrRS were immunoquantified using anti-TyrRS antibody.

FIG. 8G presents primary cortical neurons treated with bafilomycin (100 nM) for up to 2 hr and the changes in the protein levels of TyrRS were immunoquantified using anti-TyrRS antibody.

FIG. 8H presents primary cortical neurons treated with CHX (400 µM) for up to 4 hr and the changes in the protein levels of TyrRS were immunoquantified using anti-TyrRS antibody.

FIG. 9A presents rat cortical neurons (DIV 9) treated with NMDA (50 µM for 5 min) and then with trans-RSV (5-50 µM) for 24 hr. Cells were then exposed to NMDA (500 µM for 5 min) and viability was assessed using MTT assay after 24 hr.

FIG. 9B presents rat cortical neurons (DIV 9) treated with NMDA (50 µM for 5 min) and then with cis-RSV (5-50 µM) for 24 hr. Cells were then exposed to NMDA (500 µM for 5 min) and viability was assessed using MTT assay after 24 hr.

FIG. 9C presents rat cortical neurons (DIV 7) transfected with TyrRS or control siRNA (75 nM) and then treated with cis-RSV (50 µM) or trans-RSV (5, 10, 50 µM) for 24 hr. Neurons were then exposed to excitotoxic NMDA (500 µM for 5 min), and viability was assessed using MTT assay after 24 hr.

FIG. 9D presents rat cortical neurons (DIV 8) treated with trans-RSV alone or combined with different doses of cis-RSV (10-50 µM) for 48 hr, and viability was measured using MTT assay.

FIG. 9E presents representative immunoblot images and quantification using specific antibodies for PARylation, PARP1, AcK16-H4, AcK56-H3 levels after treatment of cortical neurons (DIV 9) with cis- and trans-RSVfor 15 min.

FIG. 9F presents rat cortical neurons (DIV 7) were transfected with control and TyrRS siRNA followed by treatment with cis- (25 µM) and trans-RSV (5 µM) for 15 min and immunoblotting and quantification using the specific antibodies for PARylation, PARP1 and TyrRS.

FIG. 10A presents representative immunoblots and quantification from chromatin fraction of cortical neurons (DIV 9) depicting PARP1 and PAR, ARH3, TyrRS after treatment with cis- and trans-RSV (50 µM) for 1 hr.

FIG. 10B presents representative immunoblots and quantification for ARH3 using anti-ARH3 antibody in the hippocampal region of AD patients (n=5) with age and sex-matched controls (n=5).

FIG. 10C presents immunoprecipitated (IP) TyrRS from cortical neurons (DIV 9) immunoblotted (IB) using anti-TyrRS and anti-ARH3 antibodies to detect the interaction of TyrRS with ARH3.

FIG. 10D presents rat cortical neurons (DIV 7) transfected with siRNA against PARP1 (siRNAPARP1) or control siRNA (75 nM) and then treated with cis-RSV (50 µM) or trans-RSV (50 µM) for 72 hr. Neuronal viability was assessed and quantified using an MTT assay. The knockdown was verified using immunoblot and quantified using specific antibodies for PARP1.

FIG. 10E presents cortical neurons (DIV 9/10) were treated with cis-or trans-RSV (50 µM) for 8 hr followed by a 30 min pulse labeling using 50 µM of nucleoside analog, CldU (5-chloro-2′-deoxyuridine). DNA fiber assay was performed according to the published protocol followed by immunostaining for single-stranded (ss) DNA and CldU.

FIG. 11A presents rat cortical neurons (DIV 9) treated with NMDA (50 µM for 5 min), AG-14361 (AG, 10 µM),olaparib (Ola, 10 µM) either alone or in combination with cis-RSV (50 µM) for 24 hr. Cells were then exposed to NMDA (500 µM for 5 min) and viability was assessed using MTT assay after 24 hr.

FIG. 11B presents rat cortical neurons (DIV 9) treated with olaparib (10 µM), AG-14361 (10 µM) and talazoparib (2 µM) either alone or in combination with trans-RSV (50 µM) for 72 hr.

FIG. 11C presents immunostaining images (scale bar, 10 µm) for v-H2AX foci (DAPI - nuclear marker) in cortical neurons (DIV 10) after treatment with olaparib (10 µM), AG-14361 (10 µM) and talazoparib (2 µM) for 24 hr.

FIG. 11D presents representative images (scale bar, 20 µm) for cortical neurons following olaparib (10 µM),AG-14361 (10 µM) and talazoparib (1 µM) treatment for 24 hr (MAP2 - neurite marker, and DAPI - nuclear marker).

FIG. 11E illustrates a proposed mechanism of cis-RSV-mediated neuroprotection and trans-RSV-mediated neurotoxicity.

FIG. 12A illustrates the mechanism of regulation of protein synthesis by the phosphorylation of eIF2α and eEF2.

FIG. 12B presents primary cortical neurons treated with BDNF (50 nM) for 1 hr alone and subjected to either immunofluorescence analysis using anti-TyrRS antibody to detect changes in the protein levels of TyrRS.

FIG. 12C presents primary cortical neurons treated with BDNF (50 nM) for 1 hr in combination with rapamycin (100 nM) and subjected to Western Blot analysis using anti-TyrRS antibody to detect changes in the protein levels of TyrRS.

FIG. 12D presents primary cortical neurons treated with DA (200 µM) for 2 hr and subjected to immunofluorescence analysis using anti-TyrRS antibody to detect changes in the protein levels of TyrRS.

FIG. 13A presents primary cortical neurons treated with cis- and trans-RSV (5-50 µM) for 2 hr and p-eIF2α were detected by Western Blot analysis using anti-p-eIF2α antibody.

FIG. 13B presents primary cortical neurons treated with cis- and trans-RSV (25 µM) for up to 8 hr and changes p-eIF2α were detected by Western Blot analysis using anti-p-elF2α antibody.

FIG. 14A presents primary cortical neurons treated with nelfinavir (20-40 µM) for 8 hr and changes in the levels of TyrRS, PheRSβ and p-eEF2 were detected by Western Blot analysis using their corresponding antibodies.

FIG. 14B presents primary cortical neurons treated with nelfinavir (20 µM) alone or in combination with cis-RSVfor 8 hr, and TyrRS was detected by Western Blot analysis using anti-TyrRS antibody.

FIG. 14C presents representative spectral images (scale bar, 20 µm) for neuronal TyrRS after treatment with neurotoxic agents (50 µM NMDA or 100 µM MPP+) for 4 hr in combination with cis- and trans-RSV (50 µM) in rat cortical neurons (DIV9).

FIG. 14D presents quantification for neuronal TyrRS after treatment with neurotoxic agents (50 µM NMDA or 100 µM MPP+) for 4 hr in combination with cis- and trans-RSV (50 µM) in rat cortical neurons (DIV9).

FIG. 15A presents primary cortical neurons were treated with ISRIB (≤50 nM) for 8 hr and changes in the levels of TyrRS, PheRSβ, p-eEF2, eEF2, p-eIF2α, and eIF2α were detected and quantified by Western Blot analysis using their respective antibodies.

FIG. 15B presents primary cortical neurons were treated with trans-RSV (25 µM) alone or in combination with ISRIB (10 nM) for 8 hr and changes in the levels of TyrRS were detected by Western Blot analysis.

FIG. 15C presents primary cortical neurons treated with ISRIB (100-500 nM) for 8 hr and changes in the levels of TyrRS, and PheRSβ were detected by Western Blot analysis using their respective antibodies.

FIG. 16A presents Immunostaining images (scale bar, 10 µm) for DNA damage marker, pSer139-H2AX foci (y-h2AX; DAPI - nuclear marker) in cortical neurons (DIV9) after pretreatment with Tyr (500 µM) for 12 hr, followed by the addition of cis-RSV (50 µM) for another 4 hr.

FIG. 16B presents primary cortical neurons treated with either Tyr (200 µM) alone or in combination with cis-RSV (50 µM) for 8 hr, and the levels of OGG1 were detected by Western Blot analysis using anti-OGG1 antibody.

FIG. 16C presents primary cortical neurons treated with D-Tyr (2.5 and 5 mM) for 8 hr, and the levels of TyrRS, and PheRSβ were detected by Western Blot analysis using their specific antibodies.

FIG. 16D presents rat cortical neurons (DIV9) treated with either D-Tyr or D-Phe or D-Trp (2 mM) for 48 hr, and viability was assessed using MTT assay.

FIG. 17A presents rat cortical neurons (DIV 9) were exposed to 5 µM etoposide (ETO) for 24 hr after pretreatment with cis-RSV or trans-RSV (50 µM) for 16 hr. Cell viability was assessed using MTT assay.

FIG. 17B presents rat cortical neurons (DIV 9) were exposed to 400 µM H₂O₂ for 24 hr after pretreatment with cis-RSV or trans-RSV (50 µM) for 16 hr. Cell viability was assessed using MTT assay.

FIG. 17C presents rat cortical neurons (DIV 9) were exposed to 10 µM MPP+ for 24 hr after pretreatment with cis-RSV or trans-RSV (50 µM) for 16 hr. Cell viability was assessed using MTT assay.

FIG. 17D presents immunoblots showing the levels of cleaved caspase-3 in rat cortical neurons (DIV 9/10) after the treatment with cis- and trans-RSV (25 and 50 µM) for 24 hr.

FIG. 17E presents immunoblots showing the levels of cleaved caspase-3 in rat cortical neurons (DIV 9/10) after the treatment with D-Tyr (2 mM) for 16 hr.

FIG. 17F presents rat cortical neurons (DIV 8) treated with trans-RSV alone or in combination with ISRIB (10 nM) for 48 hr and viability was measured using MTT assay.

FIG. 17G presents rat cortical neurons (DIV 8) were treated with trans-RSV alone or in combination with A484954 (100 nM) for 48 hr and viability was measured using MTT assay.

FIG. 17H presents rat cortical neurons (DIV 8) were treated with trans-RSV alone or in combination with DA (100 mM) for 72 hr and viability was measured using MTT assay.

FIG. 17I presents quantification of immunoblot images for AcK16-H4.

FIG. 17J presents quantification of immunoblot images for AcK56-H3.

FIG. 17K presents quantification of TyrRS protein levels.

FIG. 18A presents representative immunoblots and quantification of the interaction of H3 with PARP1. Cortical neurons (DIV 9) were treated with cis- and trans-RSV (5-50 µM) for 30 min and PARP1 was immunoprecipitated (IP) using antibody against PARP1. The interaction of PARP1 with H3 was determined using anti-H3 antibody.

FIG. 18B presents representative immunoblots using specific antibodies, and quantification from chromatin fraction of cortical neurons (DIV 9) depicting HPF1, OGG1, and FEN1 after treatment with cis- and trans-RSV (50 µM) for 1 hr.

FIG. 18C presents the quantification of neuronal DNA fibers.

FIG. 19Apresents monosome formation by rapamycin in rat cortical neurons.

FIG. 19B presents monosome formation by cis-RSV in rat cortical neurons.

FIG. 19C presents cis-RSV-mediated recruitment of raptor to Rab7a-coated late endosomes.

FIG. 20A presents monosome formation by physiological amyloid beta 42 (10 picomolar) in rat cortical neurons which is lost in combination with PARP1 inhibitor talazoparib.

FIG. 20B presents western blots showing activation of histone serine-ADP-ribosylation by picomolar concentration of amyloid beta 40 and 42 peptides.

FIG. 20C presents western blots showing stimulation of neuronal TyrRS protein levels by picomolar concentration of amyloid beta 40 and 42 peptides in a protein synthesis and transcription-dependent manner.

FIG. 20D presents western blots showing trapping of auto-PARylated PARP1 along with Rad52, DNA polymerase beta, ligase III, TyrRS onto the chromatin by picomolar concentration of amyloid beta 42 peptide.

FIG. 20E presents western blots showing tyrosine-mediated trapping of PARP1 through the inhibition of auto-PARylation potentially by depleting chromatin associated TyrRS.

FIG. 21 presents quantification of DNA damage using comet assay after treatment with L-tyrosine and L-phenylalanine for 16 hr and/or amyloid beta peptides for 2 hr either alone or in combination.

FIG. 22 presents quantification of DNA repair by measuring the amount of CldU incorporated into neuronal DNA after treatment with L-tyrosine and L-phenylalanine for 16 hr and/or amyloid beta peptides for 4 hr either alone or in combination.

Repeat use of reference characters in the present specification and figures is intended to represent the same or analogous features or elements of the present invention.

### DETAILED DESCRIPTION

It is to be understood by one of ordinary skill in the art that the present discussion is a description of exemplary embodiments only and is not intended as limiting the broader aspects of the present disclosure.

In general, the present disclosure is directed to compositions and methods for ‘trapping’ PARP1 to selectively modulate serine-(poly/mono)-ADP-ribosylation-dependent single strand DNA break repair (SSBR) and/or double strand DNA break repair (DSBR) along with transcription and protein synthesis by regulating the cellular localization and de novo synthesis of TyrRS. In embodiments, the present disclosure is directed to methods and compositions for use in inhibiting, modulating and/or treating a disorder in a subject (e.g., human or non-human).

Composition disclosed herein can include a tyrosine derivative compound of Formula I:

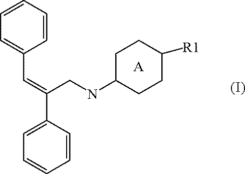

or a pharmaceutically acceptable salt and their isomers thereof, wherein ring A substituted or unsubstituted carbocyclyl, substituted or unsubstituted heterocyclyl, substituted or unsubstituted aryl, substituted or unsubstituted heteroaryl; and R1 is

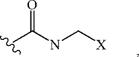

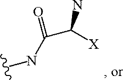

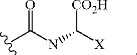

In another embodiment, composition disclosed herein can include a compound of Formula II:

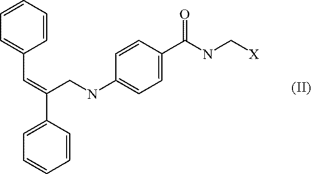

or a pharmaceutically acceptable salt and their isomers thereof, wherein R is as defined for Formula I.

In yet another embodiment, composition disclosed herein can include a compound of Formula III:

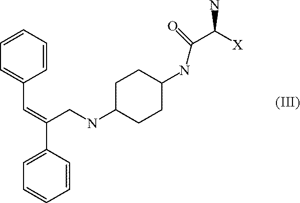

or a pharmaceutically acceptable salt and their isomers thereof, wherein R is as defined for Formula I.

In another embodiment, composition disclosed herein can include a compound of Formula IV:

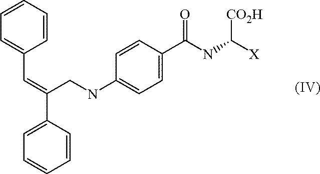

or a pharmaceutically acceptable salt and their isomers thereof.

In various embodiments, X is an amino acid side chain, —H, —CH₃, —CH₂SeH, —CH₂CO₂H, —CH₂OH, —CH(CH₃)OH, —CH₂CH₂CO₂H, —CH₂CH₂CONH₂,

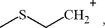

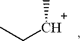

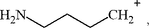

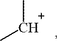

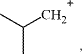

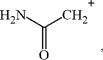

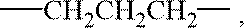

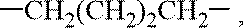

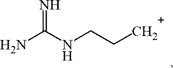

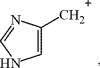

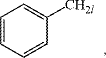

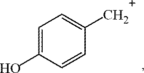

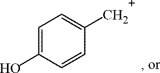

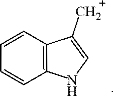

In yet another embodiment, composition disclosed herein can include a compound of Formula (V):

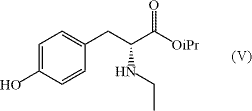 [SMILES: CCN[C@H](Cc1ccc(O)cc1)C(=O)OC(C)C]

or a pharmaceutically acceptable salt and their isomers thereof. “OiPr” as used herein refers to an isopropoxide group.

In yet another embodiment, composition disclosed herein can include a compound of Formula (VI):

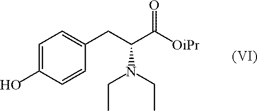 [SMILES: CCN(CC)[C@H](Cc1ccc(O)cc1)C(=O)OC(C)C]

or a pharmaceutically acceptable salt and their isomers thereof.

The term “amino acid” as used herein refers to naturally occurring amino acids, non-naturally occurring amino acids, and amino acid analogs. Naturally occurring amino acids include, for instance, the 20 (L)-amino acids commonly utilized during protein biosynthesis and 20 (D)-amino acids not utilized for protein biosynthesis and selenocysteine, and other plant-derived non-proteogenic amino acids such as pyrrolysine. Non-naturally occurring amino acids include, for instance, pomaglumetad (LY404039). Amino acid analogs may include modified forms of naturally or non-naturally occurring amino acids, for instance, substitution or replacement of chemical groups and moieties on the amino acid or by derivatization of the amino acid.

In one embodiment, the amino acid side chain may be from an acidic amino acid. For example, the amino acid residue has a negative charge due to loss of hydrogen ion at physiological pH. Amino acids having an acidic side chain may include glutamic acid and aspartic acid.

The amino acid side chain may be from a basic amino acid residue. For example, the residue may have a positive charge due to association with hydrogen ions at physiological pH or within one or two pH units thereof. Amino acids having a basic side chain include arginine, lysine, and histidine.

The amino acid side chain may be from a hydrophobic amino acid residue. For example, the residue is not charged at physiological pH. Amino acids having a hydrophobic side chain include tyrosine, valine, isoleucine, leucine, methionine, phenylalanine, and tryptophan.

The amino acid side chain may be from a neutral/polar amino acid residue. For instance, the residue is not charged at physiological pH and the residue is not sufficiently repelled by aqueous solution so that it would seek inner positions in the conformation of a peptide or protein in which it is contained when the peptide is in an aqueous medium. Amino acids having a neutral/polar side chain include asparagine, glutamine, cysteine, histidine, serine, and threonine.

Tyrosyl-tRNA synthetase (TyrRS) belongs to a family of aminoacyl-tRNA synthetases (aaRSs) that activates aromatic amino acids, such as tyrosine, for protein synthesis. As described above, tyrosine exists as enantiomers (L- and D-tyrosine), both of which are activated by TyrRS. Only naturally occurring L-tyrosine is utilized for protein synthesis. Similarly, only L-tyrosine, not D-tyrosine, acts as a substrate for tyrosine hydroxylase (TH) that catalyzes the rate-limiting step in the synthesis of dopamine. Increased tyrosine levels may modulate TyrRS. For instance, increased tyrosine levels decrease TyrRS and cause neuronal oxidative DNA damage by the simultaneous inhibition of both protein synthesis and DNA repair. Also, tyrosine inhibits TyrRS-mediated activation of poly-ADP-ribose polymerase (PARP1), a modulator of DNA repair.

Composition disclosed herein can also include one or more tyrosine or resveratrol derivatives. Resveratrol has a tyrosine-like phenolic ring that mimics tyrosine in binding to the active site of TyrRS. Resveratrol (RSV) exists as a mixture of two isomers, cis-RSV and trans-RSV. Interestingly, recent studies demonstrated that the sulfate metabolites of trans-RSV provide an intracellular pool to generate cis-RSV. Further, analysis of the x-ray crystal structures of TyrRS with and without cis-RSV showed that the binding of cis-RSV in the active site of TyrRS mimics its ‘tyrosine-free’ conformation. Thus, binding of cis-RSV may enable the moonlighting functions of TyrRS even in the presence of tyrosine. For example, D-tyrosine and trans-RSV, upon binding to TyrRS and mimicking a ‘tyrosine-like’ conformation, decrease TyrRS, inhibits DNA repair, and cause neurotoxicity by inhibiting HPF1/PARP1-dependent serine-ADP-ribosylation. Conversely, cis-RSV, upon binding to TyrRS and mimicking a ‘tyrosine-free’ conformation, increases TyrRS, facilitates DNA repair through serine-ADP-ribosylation, and provides neuroprotection in a TyrRS-dependent manner. Thus, increased tyrosine and/or phenylalanine levels may have causal effects in aging and age-associated diseases. Thus, derivatives of tyrosine/phenylalanine and resveratrol that modulate serine-(poly/mono)-ADP-ribosylation will have tremendous therapeutic potential against aging and age-associated diseases.

Moonlighting functions of TyrRS are responsible for activating the auto-poly-ADP-ribos(PAR)ylation of poly-ADP-ribose polymerase 1 (PARP1) and associated stress signaling. Consistently, auto-PARylation of PARP1 is circadian-regulated in a feeding-dependent manner in which feeding that increases tyrosine levels inhibits auto-PARylation. These observations suggest that the moonlighting functions of TyrRS are normally activated when tyrosine levels are decreased during the nadir/trough of circadian rhythm.

Disclosed herein are methods of modulating levels of tyrosine or TyrRS in a subject utilizing tyrosine or RSV derivatives as described herein. The term “modulating” refers to increasing, enhancing, stimulating, decreasing, or reducing in a statistically or a physiologically significant amount relative to a control. For instance, the level of a subject is increased relative to a control amount by more than about 10% or more, such as more than about 20% or more, such as more than about 30% or more, such as more than about 40% or more, such as more than about 50% or more, or such as more than about 60% or more. Contrarily, the level of a subject, for instance, is decreased relative to a control amount by more than about 10% or more, such as more than about 20% or more, such as more than about 30% or more, such as more than about 40% or more, such as more than about 50% or more, or such as more than about 60% or more. Modulatory methods disclosed herein may involve contacting a cell or tissue with a compound that inhibits or reduce the expression and/or activity of tyrosine or TyrRS. For example, D-tyrosine or cis-RSV compounds may be administered in vitro or ex vivo (e.g., by contacting the cell with such compounds) or, alternatively, in vivo (e.g., administering the compound to a subject).

The present disclosure contemplates methods of treating a disorder in a subject comprising administering a compound disclosed herein or a pharmaceutically acceptable salt thereof. In one embodiment, the disorder is mediated by tyrosine and/or TyrRS. For example, the disorder is a neurocognitive disorder and/or sleep disorder such as insomnia, hypersomnia, frontotemporal dementia, Alzheimer’s disease, Parkinson’s disease, amyotrophic lateral sclerosis, multiple sclerosis, Huntington’s disease, epilepsy and seizures, learning disabilities, neuromuscular disorders, Cockayne syndrome, cerebral palsy, dystonia, spinocerebellar ataxia with axonal neuropathy-1 (SCAN1), Angelman Syndrome, COVID-19-related neurocognitive problems, chemotherapy-associated neurocognitive problems including ‘chemo brain’, autism spectrum disorder (ASD), delirium, mild-cognitive impairment, traumatic brain injury, phenylketonuria, or tyrosinemia. In another embodiment, the disorder is a metabolic disorder such as heart failure, cardiovascular disease, autoimmune-related disorders, myocardial ischemia reperfusion injury, hypertension, stroke, septic encephalopathy, diabetes, obesity, sepsis, Systemic Lupus Erythematosus or inflammation. Similarly, in other embodiment, the disorder is cancerous growth of tissues or cells in different parts of the body, such as breast cancer, ovarian cancer, colon cancer, pancreatic cancer, lung cancer, prostate cancer, brain tumor, leukemia, bone cancer, and cachexia.

According to one embodiment, a composition disclosed herein and/or a pharmaceutically compatible carrier can be delivered to the targeted cells or tissue via any pharmaceutically acceptable delivery system. The term “administering” is intended to include modes and routes of administration which allow a compound to perform its intended function. In general, the compounds disclosed herein may be administered to a subject according to known methods, including injection (subcutaneous, intravenous, parenterally, intraperitoneally, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The compound may be administered alone, or in conjunction with a pharmaceutically acceptable carrier. The compound also may be administered as a prodrug, which is converted to its active form in vivo.

Pharmaceutically acceptable carriers include, but are not limited to, saline, buffered saline, glucose in saline, etc. Solid supports, liposomes, nanoparticles, microparticles, nanospheres or microspheres may also be used as carriers for administration of a compound disclosed herein. As used herein, the term “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, dispersion media, coatings, antibacterial or antifungal agents, isotonic and absorption delaying agents, and the like, compatible with pharmaceutical administration. The use of such media and agents for pharmaceutically active substances is well-known in the art. Supplementary agents can also be incorporated into the compositions.

The therapeutically effective amount of the composition can vary based on factors such as the disorder stage, age, sex, and weight of the individual, and the ability of the compound to elicit a desired response in the subject. Further, compounds disclosed herein can be administered to a subject at one time or over a series of treatments and may be administered to the subject at any time.

In one embodiment, a therapeutically effective dosage of a compound can be administered at a concentration from about 1 micromolar (µM) to about 100 µM, such as from about 2 µM to about 95 µM, such as from about 10 µM to about 85 µM, such as from about 20 µM to about 75 µM, such as from about 35 µM to about 50 µM, or any range therebetween. As expected, the dosage will be dependent on the condition, size, and age of the subject.

Compounds disclosed herein may be administered, as appropriate or indicated, in a single dose as a bolus or by continuous infusion, or as multiple doses by bolus or by continuous infusion. Multiple doses may be administered, for example, multiple times per day, once daily, multiple times per week, every 2, 3, 4, 5, 6 or 7 days, weekly, every 2, 3, 4, 5 or 6 weeks, or monthly. However, other dosage regimens may be useful. The progress of this therapy is easily monitored by conventional techniques.

It can be advantageous to formulate oral or parenteral compositions in dosage unit form for ease of administration and uniformity of dosage. Dosage unit form as used herein includes physically discrete units suited as unitary dosages for the subject to be treated; each unit may contain a predetermined quantity of active compound calculated to produce the desired therapeutic effect in association with the required pharmaceutical carrier. The specification for the dosage unit forms of the application is dictated by and directly dependent on the unique characteristics of the active compound and the particular therapeutic effect to be achieved, and the limitations inherent in the art of compounding such an active compound for the treatment of individuals.

Pharmaceutical compositions for parenteral, intradermal, or subcutaneous injection can include pharmaceutically acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include, but are not limited to, water, ethanol, polyols (e.g., glycerol, propylene glycol, polyethylene glycol and the like), carboxymethylcellulose and suitable mixtures thereof, vegetable oils (e.g., olive oil), and injectable organic esters such as ethyl oleate. A composition can contain minor amounts of auxiliary substances such as wetting or emulsifying agents, pH buffering agents and the like that can enhance the effectiveness of the active ingredient. Proper fluidity may be maintained, for example, by the use of coating materials such as lecithin, by the maintenance of the required particle size in the case of dispersions, and by the use of surfactants. A composition may also contain adjuvants such as preservatives, wetting agents, emulsifying agents, and dispersing agents. It may also be desirable to include isotonic agents such as sugars, sodium chloride and the like.

For intravenous administration, suitable carriers include, without limitation, physiological saline, bacteriostatic water, Cremophor EL™ (BASF™, Parsippany, N.J.) or phosphate buffered saline (PBS). In all cases, an injectable composition should be sterile and should be fluid to the extent that easy syringeability exists. It must be stable under the conditions of manufacture and storage and must be preserved against the contaminating action of microorganisms such as bacteria and fungi. Prevention of the action of microorganisms may be ensured by the inclusion of various antibacterial and antifungal agents such as paraben, chlorobutanol, phenol, sorbic acid and the like. Prolonged absorption of the injectable compositions can be brought about by including in the composition an agent that delays absorption, for example, aluminum monostearate or gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents and/or adjuvant materials can be included as part of an orally ingestible composition. The tablets, pills, capsules, troches and the like can contain any of the following ingredients or compounds of a similar nature: a binder such as microcrystalline cellulose, gum tragacanth, or gelatin; an excipient such as starch or lactose; a disintegrating agent such as alginic acid, Primogel®, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

When administered orally in liquid form, a liquid carrier such as water, petroleum, oils of animal or plant origin (e.g., peanut oil, mineral oil, soybean oil, or sesame oil), or synthetic oils may be added. A liquid form may further contain physiological saline solution, dextrose or other saccharide solution, or glycols such as ethylene glycol, propylene glycol, or polyethylene glycol. When administered in liquid form, a composition can contain from about 0.5 to 90% by weight of a D-tyrosine or trans-RSV derivative.

For administration by inhalation, the D-tyrosine or trans-RSV derivative can be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, a pharmaceutical composition can be formulated for sustained or controlled release of the compound (e.g., D-tyrosine or trans-RSV derivative). Biodegradable, biocompatible polymers, such as ethylene vinyl acetate, polyanhydrides, polyglycolic acid, collagen, polyorthoesters, and polylactic acid can be used. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art.

It is to be understood that the in vivo methods have application for both human and veterinary use. The methods of the present invention contemplate single, as well as multiple, administration, given either simultaneously or over an extended period of time.

The present disclosure may be better understood with reference to the following examples.

### EXAMPLES

### Materials and Methods

### Cell Culture

Primary cortical neurons were dissected from E18 Sprague Dawley rat pups in Hibernate E (BrainBits®) and dissociated using the Neural Tissue Dissociation kit (Miltenyi Biotec). Minced cortices were incubated in a pre-warmed enzyme mix at 37° C. for 15 min; tissues were then triturated and strained using a 40 µm cell strainer. After washing and centrifugation, neurons were seeded in 50 µg/ml poly-D-Lysine (Sigma-Aldrich) coated tissue culture plates. NBActive-1 medium (BrainBits®) supplemented with 100 U/ml of Penicillin-Streptomycin (Life Technologies™), 2 mM L-Glutamine (Life Technologies™), and 1X N21 supplement (R&D Systems™) was used as culture medium. For preparation of culture medium containing reduced tyrosine, the components of NBActive-1 medium were altered to combined with a reduced concentration of tyrosine, as mentioned in Table 1, and was supplemented with 100 U/ml of Penicillin-Streptomycin (Life Technologies™), 2 mM L-Glutamine (Life Technologies™), and 1X N21 supplement (R&D Systems™) to obtained culture medium with reduced tyrosine. Control (non-targeting), TyrRS, and PARP1 siRNAs were obtained from Invitrogen™ (# AM4635, s443, and s130207, respectively). Rat cortical neurons at 5 DIV were transfected with 75 nM control or TyrRS siRNA using DharmaFECT™ 3 Transfection Reagent. A second transfection was done two days later using 75 nM of TyrRS siRNA, followed by cell collection or assays after another 48 hr. For PARP1 siRNA, neurons at 7 DIV were transfected with 75 nM siRNA for both control and PARP1 siRNA.

TABLE 1

Composition for low-tyrosine medium

| Component | Conc. (mM) |
|---|---|
| Glycine | 0.4 |
| L-Alanine | 0.022 |
| L-Arginine hydrochloride | 0.398 |
| L-Asparagine-H₂O | 0.005 |
| L-Cysteine | 0.26 |
| L-Histidine hydrochloride-H₂O | 0.2 |
| L-Isoleucine | 0.801 |
| L-Leucine | 0.801 |
| L-Lysine hydrochloride | 0.797 |
| L-Methionine | 0.201 |
| L-Phenylalanine | 0.4 |
| L-Proline | 0.067 |
| L-Serine | 0.4 |
| L-Threonine | 0.798 |
| L-Tryptophan | 0.078 |
| L-Tyrosine | 0.2 |
| L-Valine | 0.803 |
| Calcium Chloride (CaCl₂) (anhydrous) | 1.8 |
| Magnesium Chloride (anhydrous) | 0.813 |
| Potassium Chloride (KCl) | 5.33 |
| Sodium Bicarbonate (NaHCO₃) | 26.19 |
| Sodium Chloride (NaCl) | 68.96 |
| Sodium Phosphate monobasic (NaH₂PO₄-H₂O) | 0.905 |
| Zinc sulfate (ZnSO₄-⁷H₂O) | 0.0006 |
| D-Glucose (Dextrose) | 25 |
| HEPES | 10.92 |
| Sodium Pyruvate | 0.227 |

### Western Blotting

Cultured neurons (DIV 9-10) were washed once with cold 1× PBS and lysed in cell lysis buffer (20 mM Tris-HCl (pH 7.5), 150 mM NaCl, 1 mM Na2EDTA, 1 mM EGTA, 1% Triton™, 2.5 mM sodium pyrophosphate, 1 mM beta-glycerophosphate, 1 mM Na₃VO₄, 1 µg/ml leupeptin supplemented with protease inhibitor). The lysates were centrifuged at 10,000 RPM for 15 mins at 4° C. to separate the chromatin-bound and soluble fractions. Lysates were quantified using Bio-Rad™ Protein Assay and equal amounts of protein were loaded onto a 4 to 12% gradient gel (Invitrogen™ NuPAGE™). Protein was transferred from the gel to 0.2 µm NC membranes at 25 V for 10 mins using transfer stacks (iBlot™ 2- Invitrogen™) and blocked with 5% non-fat milk in TBST (10 mM Tris-HCl pH 8.0, 150 mM NaCl, 0.01 % Tween-20) for 1 hr before application of primary antibodies. Primary and secondary antibodies were incubated overnight at 4° C. and for 1 hr at room temperature, respectively. Immobilon® ECL Ultra Western HRP Substrate (WBULS0500, Millipore™) and a luminescent image analyzer (ChemiDoc™ Imaging System, Bio-Rad™) were used to detect proteins. Quantification of western blots was done using ImageJ (Version 1.53c).

TABLE 2

Listing of Antibodies used for Western Blotting

| Antibody | Company | Catalog No. | Dilution |
|---|---|---|---|
| Acetyl-Histone H3 (Lys56) | Cell Signaling Technology™ | 4243 | 1:1000 (WB) |
| Acetyl-Histone H3 (Lys9) | Cell Signaling Technology™ | 9649 | 1:1000 (WB) |
| ARH3 | Proteintech® | 16504-1-AP | 1:1000 (WB) |
| eEF2 | Cell Signaling Technology™ | 2332 | 1:1000 (WB) |
| elF2α | Cell Signaling Technology™ | 5324 | 1:1000 (WB) |
| Fen1 | Proteintech® | 14768-1-AP | 1:1000 (WB) |
| GAPDH | Cell Signaling Technology™ | 2118 | 1:2000 (WB) |
| H3 | Proteintech® | 17168-1-AP | 1:1000 (WB) |
| H3-S10-ADP-Ribose | Bio-RAD™ | HCA357 | 1:1000 (WB) |
| H4 | Proteintech® | 16047-1-AP | 1:1000 (WB) |
| HPF1 | Novus Biologicals™ | NBP1-93973 | 1:1000 (WB) |
| OGG1 | Proteintech® | 15125-1-AP | 1:1000 (WB) |
| PARP1 | Proteintech® | 66520-1-lg | 1:1000 (WB) |
| PARP2 | Abcam | ab177529 | 1:500 (WB) |
| PheRSα | Proteintech® | 18121-1-AP | 1:1000 (WB) |
| PheRSβ | Proteintech® | 16341-1-AP | 1:1000 (WB) |
| Phospho-eEF2 (Thr56) | Cell Signaling Technology™ | 2331 | 1:1000 (WB) |
| Phospho-elF2α (Ser51) | Cell Signaling Technology™ | 3398 | 1:1000 (WB) |
| Poly (ADP-Ribose) Polymer | Abcam | ab14459 | 1:1000 (WB) |
| PP2A C | Cell Signaling Technology™ | 2038 | 1:1000 (WB) |
| Puromycin | Millipore™ | MABE343 | 1:1000 (WB) |
| TyrRS | Abcam | ab50961 | 1:1000 (WB) |
| β-Tubulin | Cell Signaling Technology™ | 2128 | 1:2000 (WB) |

### Comet Assay

Cultured cortical neurons (DIV9) were treated with cis- or trans-RSV (50 µM), in combination with tyrosine (1 mM) for 1 hr. The cells were harvested with a cell scraper using chilled PBS and counted. The comet assay (Trevigen®, Gaithersburg, MD) was performed according to standard methods using alkaline conditions. Electrophoresis was carried out at the rate of 1.0 V/cm for 20 min. The slides were removed from the electrophoresis chamber, washed twice in deionized water for 5 min and immersed in 70% ethanol for 5 min. Subsequently, the slides were dried at 37° C. for 30 min, DNA was stained with 50 µL of SYBR™ Gold dye (Fisher Scientific, 1:10000 in Tris-EDTA buffer, pH 7.5) for 20 min in the dark at room temperature and then analyzed using an epifluorescent microscope at 10X magnification. The images were scored for comet parameters, such as tail length and tail moment (product of % of DNA in the tail and tail length), using the TriTek CometScore™ Freeware v1.5 image analysis software.

### DNA Fiber Analysis

Cultured cortical neurons (DIV9) were treated with cis- and trans-RSV (50 µM) for 8 hr, followed by the addition of thymidine analog, 50 µM CldU (5-Chloro-2′-deoxyuridine) for 30 min. Briefly, cells were isolated by trypsinization, embedded in agarose plugs, and subjected to proteinase K (0.5% SDS, 0.1 M EDTA, 1 mg/ml Proteinase K) digestion at 50° C. for 16 hr. Plugs were dissolved with agarose (Fisher [NEB], 50-811-726) for 16 hr. Molecular combing was performed using the FiberComb® Molecular Combing System (Genomic Vision) with a constant stretching factor of 2 kb/µm using vinylsilane coverslips (20 × 20 mm; Genomic Vision). Combed coverslips were incubated at 60° C. for 2 hr in a pre-warmed hybridization oven to minimize photobreaking, followed by denaturation of the DNA fibers (0.5 M NaOH + 1 M NaCl) for 8 min. The coverslips were then washed with PBS, followed by serial ethanol dehydration (70%-100%). Following two 1x PBS washes, the coverslips were blocked in 3% BSA/1x PBS for 30 min followed by incubation with α-BrdU (for CldU) (BD [347,580]) (1:100) and ssDNA antibody (Millipore™ MAB3034) (1:100), for 2 hr at 37° C. After three PBST washes, secondary antibody incubation was done using α-mouse Alexa Fluor® 594 and α-rat Alexa Fluor® 488 (1:500) for 1 hr at 37° C. Coverslips were washed three times with 1x PBST, dehydrated and mounted on slides with mounting media. The stained DNA fibers were visualized using a fluorescence microscope (EVOS FL, Thermo Fisher™ Scientific). Analysis was performed in ImageJ by counting the total ssDNA and the CldU labeled fibers. For each treatment condition, 300 fibers were counted, and the average ratio of CldU incorporation for ssDNA fibers per condition was used for final representation.

### Immunofluorescence (IF)

Cultured cortical neurons (DIV 9-10) were fixed in 4% formaldehyde for 15 min, followed by permeabilization and blocking for 30 min in 5% BSA (PBS) and 0.1% Tween™ 20 at room temperature. Incubation with primary antibodies was done at 4° C. overnight. The names and dilutions of the primary antibodies used for IF are described in Table 3. Secondary antibodies were incubated for 1 hr at room temperature. Secondary antibodies used were Alexa Fluor® 647 (anti-chicken), Alexa Fluor® 555 (anti-mouse), Alexa Fluor® 488 (anti-rabbit) from Invitrogen™ at 1:1000 dilution. Coverslips were then mounted using DAPI (4′,6-diamidino-2-phenylindole)-supplemented mounting medium, ProLong™ Gold Antifade (Invitrogen™) and imaged with Leica DMI6000 epifluorescent microscope using oil immersion 63x/NA 1.4 objective. The quantification for total protein levels in neurons was done using ImageJ (version 1.53c), and imaging parameters were matched for exposure, gain, and offset.

TABLE 3

List of antibodies used for IF

| Antibody | Company | Catalog No. | Dilution |
|---|---|---|---|
| MAP2 | Abcam | Ab5392 | 1:500 |
| phospho-histone H2AX (Ser139) | Cell Signaling Technology™ | 9178 | 1:400 |
| TyrRS | Novus Biologicals™ | NBP1-32551 | 1:200 |
| 8-hydroxy-2′-deoxyguanosine | Abcam | ab48508 | 1:200 |

### Drug Treatments

All drugs/inhibitors stock solutions (1000x) were prepared in DMSO or ethanol and diluted in culture media for final concentration. cis-RSV was purchased from Cayman Chemicals (Item No. 10004235, ≥ 98% purity) and trans-RSV was purchased from Millipore-Sigma (Catalog No. 34092, ≥ 99% purity) and the stocks were prepared in ethanol. The various compounds used for treatments and their stock concentrations in Table 4.

TABLE 4

| | | | | |
|---|---|---|---|---|
| Compound | Catalog | Stock Conc. | Final Conc. | Solvent |
| Rapamycin | 53123, Alfa Aesar™ | 100 µM | 5-50 nM | DMSO |
| ISRIB | 5284, Tocris | 250 µM | 5-500 nM | DMSO |
| NMDA | 0114, Tocris | 50 mM | 50 µM | PBS |
| MPP⁺ Iodide | D048, Sigma-Aldrich | 100 mM | 50 µM | DMSO |
| A484594 | 324516, Millipore™ | 100 µM | 100 nM | DMSO |
| Nelfinavir esylate hydrate | N0986, TCl | 100 µM | 20-40 µM | PBS |
| L-Tyr | 194759, MP Biomedicals™ | 100 mM | 0.1-0.5 mM | PBS |
| Dopamine HCl | H60255, Sigma-Aldrich | 100 mM | 0.1-0.5 mM | PBS |
| L-DOPA | A11311, Alfa Aesar™ | 100 mM | 0.1-0.5 mM | PBS |
| L-Phe | A13238, Alfa Aesar™ | 100 mM | 0.1-0.5 mM | PBS |
| D-Tyr | 143865, BTC | 100 mM | 0.5-2 mM | PBS |
| D-Trp | 215145, BTC | 100 mM | 0.1-1 mM | PBS |
| D-Phe | 225200, BTC | 100 mM | 0.5-2 mM | PBS |
| 6-OHDA (hydrobromide) | 25330, Cayman Chemical™ | 100 mM | 0.1 - 0.3 mM | PBS |
| BDNF | B3795-5UG, Sigma-Aldrich | 100 µg/ml | 100 ng/ml | PBS |

### Neurite Degeneration Index

The neurite degeneration index was calculated. Samples were imaged using ImageXpress® Micro 4 at a magnification of 10x to capture the entire field of interest. The samples analyzed for neurite degeneration were stained using the standard immunofluorescence procedure with MAP2 (Alexa Fluor® 647) for neurites and DAPI staining for the nucleus. Neurite degeneration was quantified using 5-6 regions of interest of equal sizes from each treatment condition. The analysis of neurite degeneration was done using ImageJ. The fluorescent images for MAP2 staining were binarized, so that pixel intensity of regions corresponding to neurite staining was converted to black, and all other regions were converted to white. Healthy intact neurites show a continuous tract, whereas degenerated axons have a particulate structure due to fragmentation or beading. To detect degenerated neurites, the particle analyzer module of ImageJ was used. The percentage of the area of the small fragments or particles (size = 3 - 10 µm²) to the intact neurites (size > 25 µm²) with information derived from the binary images was calculated. A degeneration index (DI) was calculated as the fragmented neurite area ratio over the intact neurite area.

### Cell Viability Assays

Rat cortical neurons (DIV9-11) were exposed to different treatments (NMDA, ETO, H₂O₂, MPP+) after seeding 20,000 cells/well in 96-well plates. Cell viability was then assessed at 48 hr after the initial exposure to NMDA. 3-[4,5-dimethylthiazole-2-yl]-2,5-diphenyltetrazolium bromide (MTT) assays were used to assess change in cell viability. Rat cortical neurons (DIV9) were exposed to 5 µM etoposide (ETO, 28435 Chem-Impex), 400 µM H₂O₂ (H1009, Sigma-Aldrich) or 10 µM MPP+ (D048, Sigma-Aldrich) for 24 hr after pretreatment with cis-RSV or trans-RSV (50 µM) for 16 hr. Cultured rat cortical neurons were incubated with MTT (0.5 mg/mL). In the MTT assay, after 2 hr incubation, the insoluble purple product formazan resulting from the reduction of MTT by NAD(P)H-dependent oxidoreductases present in cells with viable mitochondria was solubilized in dimethyl sulfoxide at room temperature, under agitation, and protected from light. The percentage of MTT reduced as measured by the difference between the absorbances at 570 nm read in a spectrophotometer (SpectraMax® 190R Molecular Devices, UK). Results are presented as a percentage of control (wells incubated with the vehicle).

### Results

### TyrRS Is Decreased in the Hippocampal Tissue Samples of Human AD Patients

AD decreases brain protein synthesis at the elongation step in humans through an unknown mechanism. Recently published human brain proteome showed decreased TyrRS and phenylalanyl-tRNA synthetase beta (PheRSβ) levels in AD-affected brain regions. Re-analysis of a second brain proteome showed that the protein levels of TyrRS and PheRSβ correlate with cognitive performance in humans. Conversely, their decrease correlates with AD status and Braak stages. Intriguingly, meta-analysis of human brain transcriptomic data from AD patients did not show any changes in the mRNA levels of TyrRS and PheRSβ. While translation of PheRSβ declines in an age-dependent manner, TyrRS levels did not correlate with any known biomarkers of AD or other neurodegenerative diseases indicating that TyrRS protein level might be modulated by hitherto unknown factors.

### Tyrosine Is Increased During Aging and in Neurocognitive and Metabolic Disorders

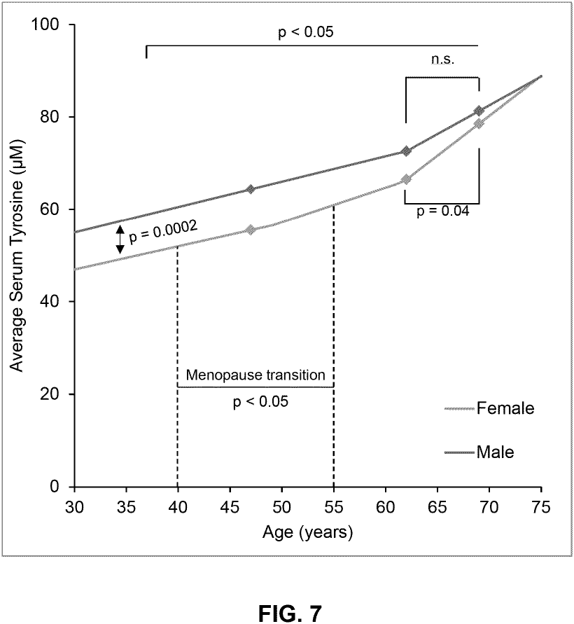

Aging is the highest risk factor for neurodegenerative diseases and intriguingly, the incidence of AD and other dementias is higher in women than in men. Tyrosine levels increase during aging and also consistently increase in AD brain tissues. Interestingly, young women have lower serum tyrosine levels than young men. However, menopause increases tyrosine levels, resulting in a significant increase in tyrosine levels in older women (FIG. 7). Beyond neurodegenerative diseases, aging in humans is associated with increased incidences of other diseases as well. Literature analysis has continuously showed that tyrosine and/or phenylalanine are increased during delirium, heart failure (HF), Parkinson’s Disease (PD), autism spectrum disorders (ASD), CVD, and other metabolic disorders in humans. Interestingly, increased levels of tyrosine and/or phenylalanine inhibit protein synthesis and induce DNA damage and promote oxidative stress in the brain in vivo in rats. Because AD decreases TyrRS levels and brain protein synthesis, it was hypothesized that increased tyrosine levels may negatively regulate TyrRS levels and cause neuronal oxidative DNA damage.

### Tyrosine Facilitates the Degradation of Neuronal TyrRS by Both Proteosome and Lysosome

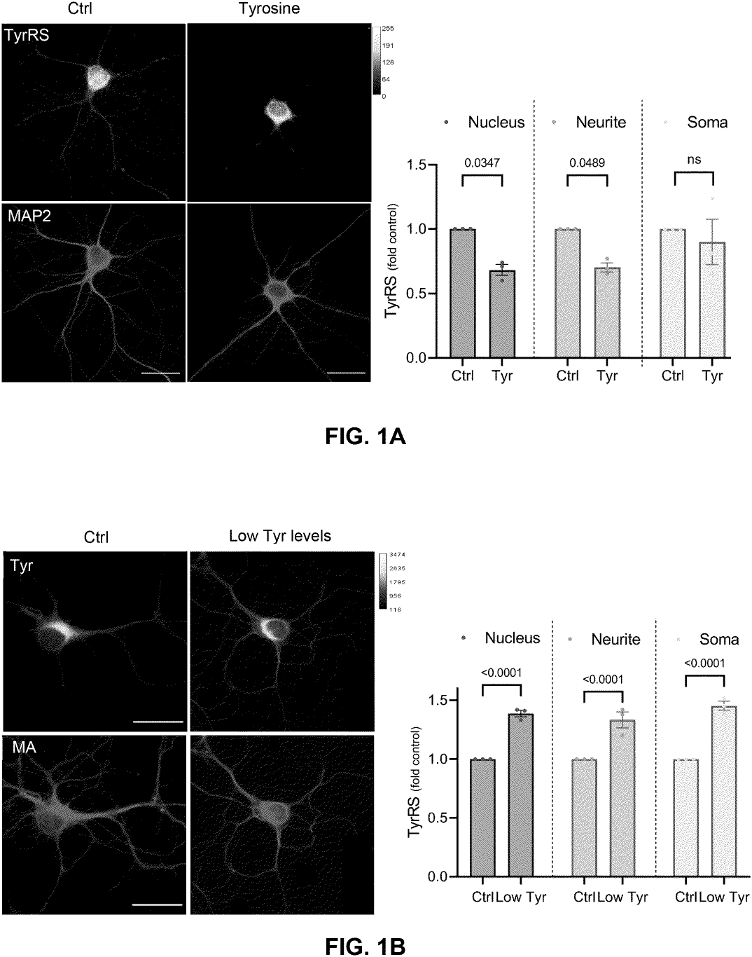

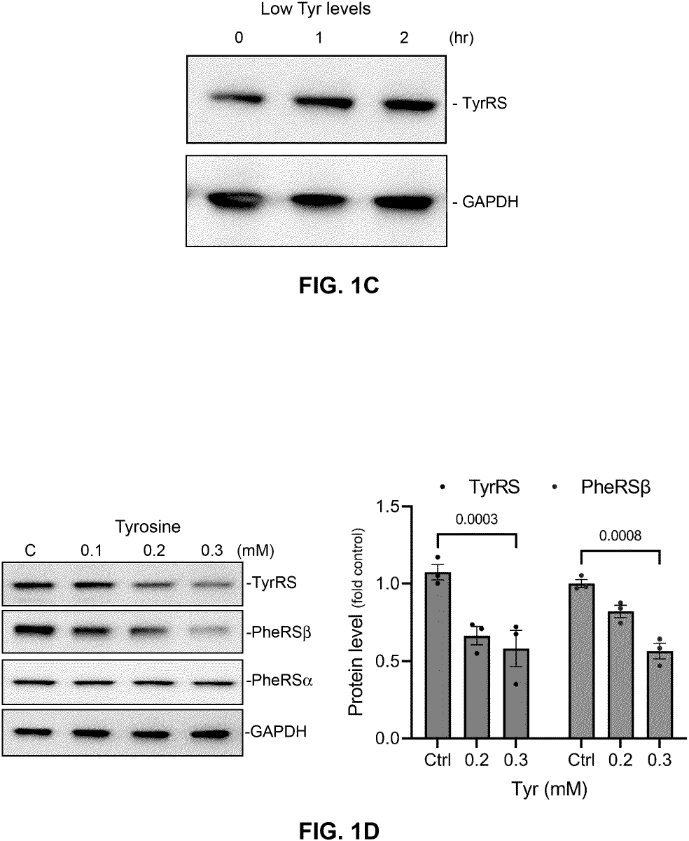

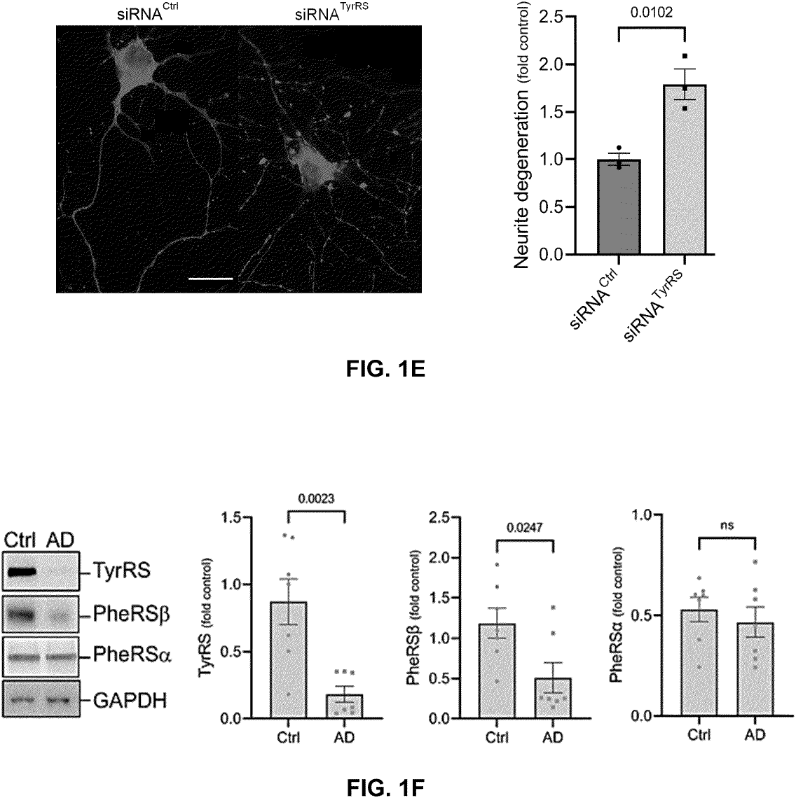

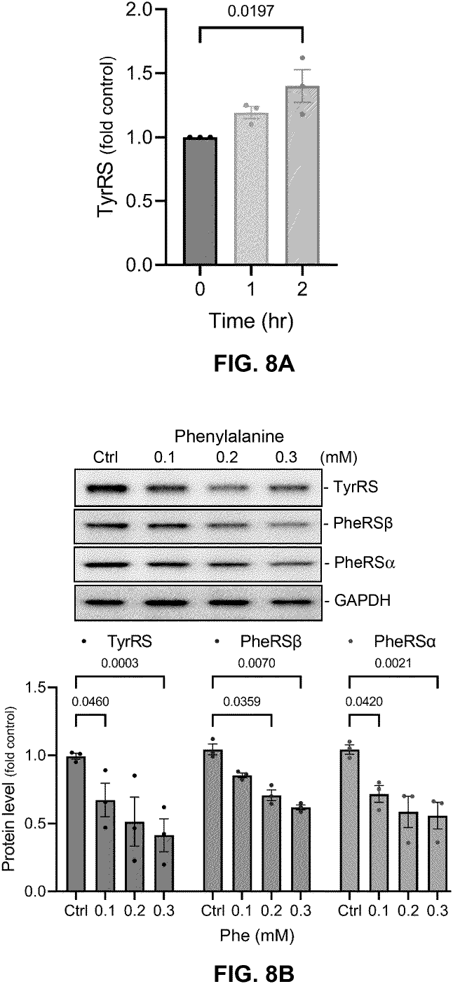

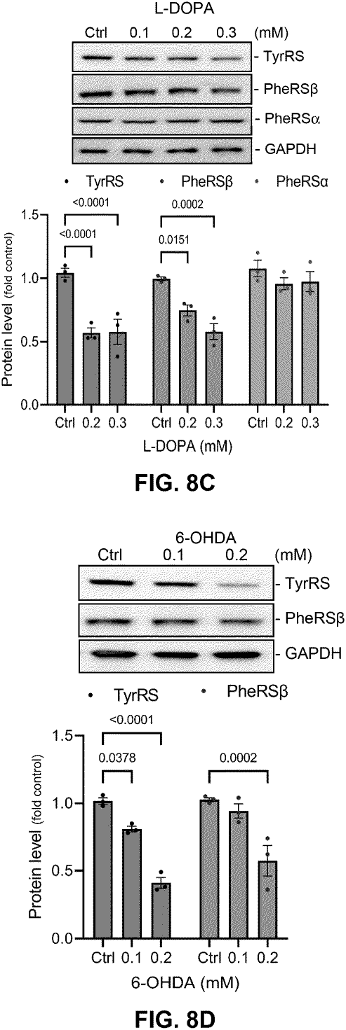

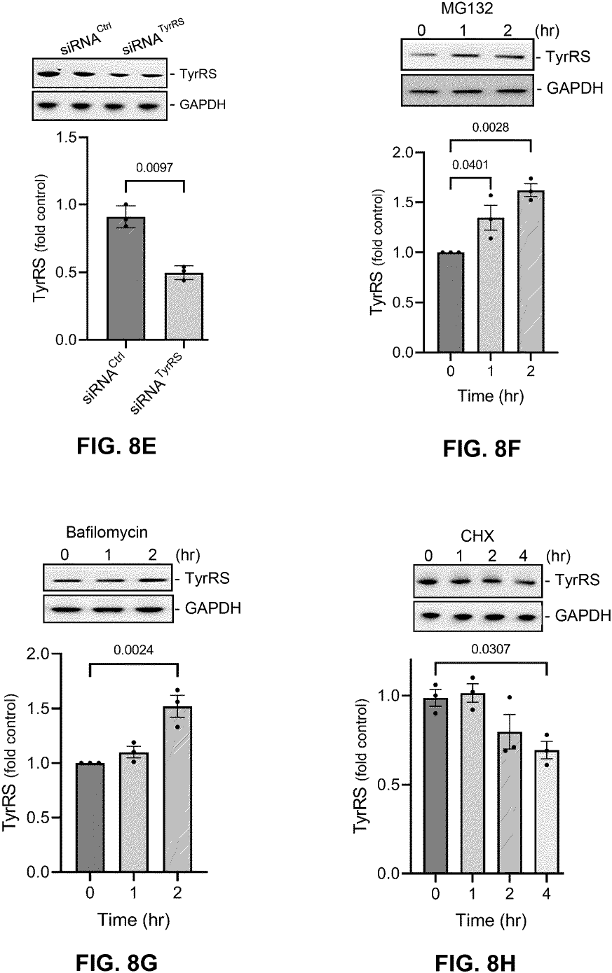

To determine whether treatment with tyrosine modulates TyrRS levels, we treated cortical neurons (DIV8/9) with increasing concentrations of tyrosine. Consistent with our hypothesis, tyrosine decreased TyrRS in rat cortical neurons (DIV 9/10) (FIG. 1A) with preferential effects in the nucleus and the neurites (FIG. 1A). Conversely, reducing tyrosine levels in the culture medium increased TyrRS levels in the nucleus and neurites (FIG. 1B). Although tyrosine depleted PheRSβ, it did not affect the levels of PheRSβ (FIG. 1E). Similarly, phenylalanine, 3,4-dihydroxy-L-phenylalanine (L-DOPA), and 6-hydroxydopamine (6-OHDA) also decreased the levels of TyrRS and PheRSβ, suggesting that derivatives of the tyrosine metabolism also negatively regulate TyrRS. Albeit an essential protein, it has been observed previously that ~75% knockdown of TyrRS using siRNA (siRNATyrRS) does not affect cell viability. However, siRNATyrRS in cortical neurons (~50% knockdown) resulted in robust neurite degeneration (FIG. 1E) indicating a critical role of TyrRS in maintaining neurite stability. Recently published mass spectrometry data showed that TyrRS is heavily ubiquitinated in the cell under stress conditions. Moreover, deubiquitination of TyrRS (along with other aaRSs) facilitates the recovery of translation during the recovery phase. To determine if tyrosine exploits the proteasomal or lysosomal pathway (autophagy) for TyrRS degradation along with its ability to inhibit protein synthesis, additional experiments were performed using inhibitors of autophagy (bafilomycin), proteasome (MG132), and protein synthesis (cycloheximide, CHX). Treatment with both bafilomycin and MG132 increased the levels of TyrRS, suggesting that both the proteasome and lysosome are involved in the constitutive degradation of TyrRS (FIG. 8E and FIG. 8F). However, cycloheximide decreased TyrRS levels (FIG. 8H), suggesting that sustained de novo synthesis is required to maintain the homeostatic levels of TyrRS under normal conditions, whereas deubiquitination may stabilize TyrRS under stress conditions to facilitate the recovery of translation.

### Dopamine Mimics BDNF in Stimulating the De Novo Synthesis of Neuronal TyrRS Protein

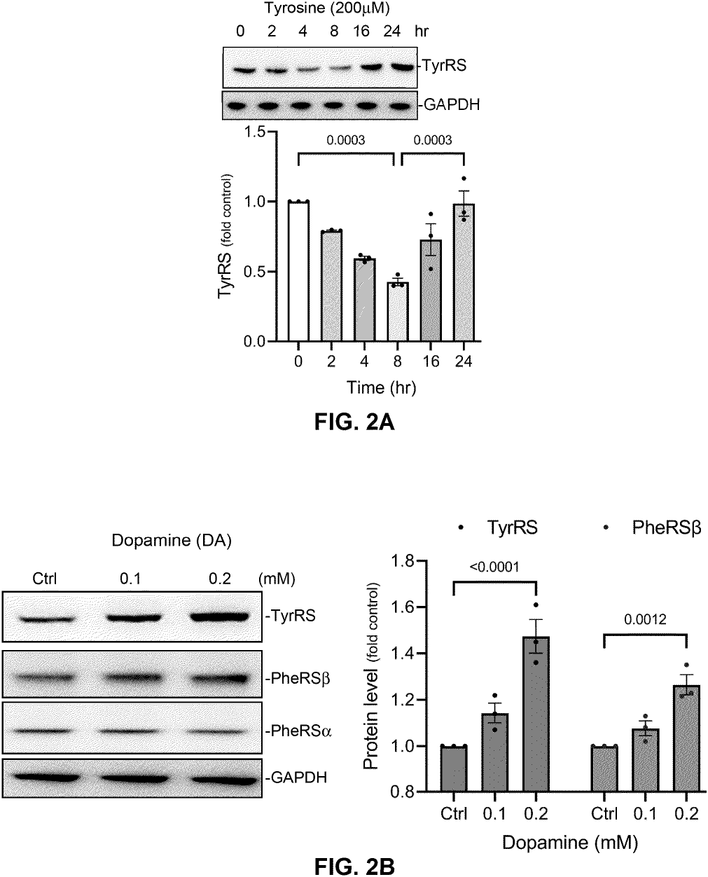

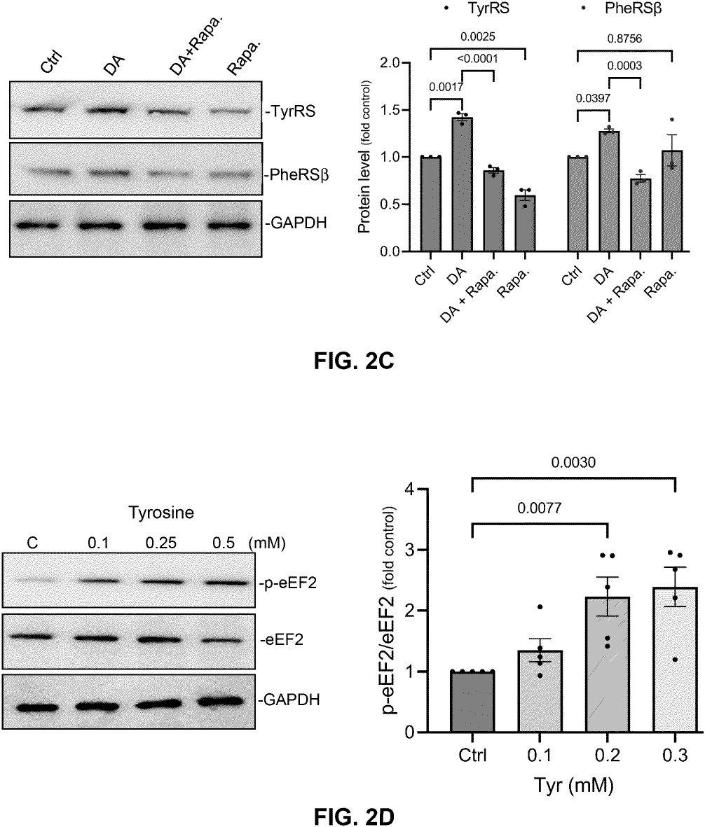

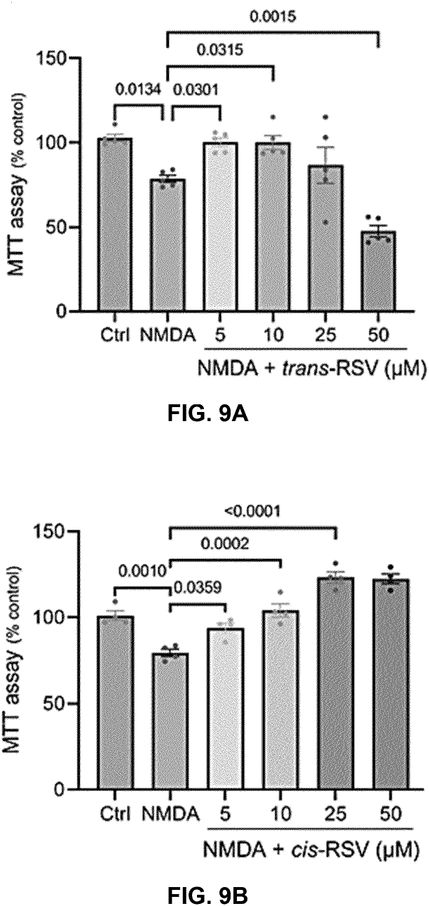

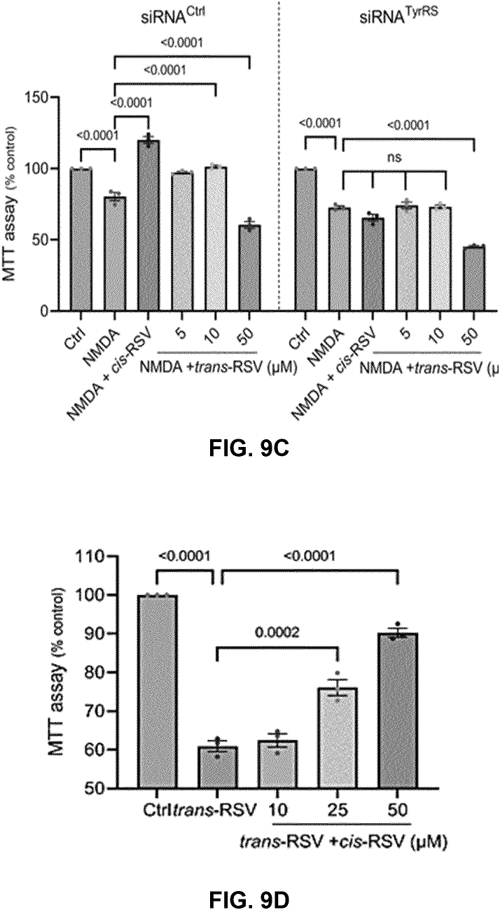

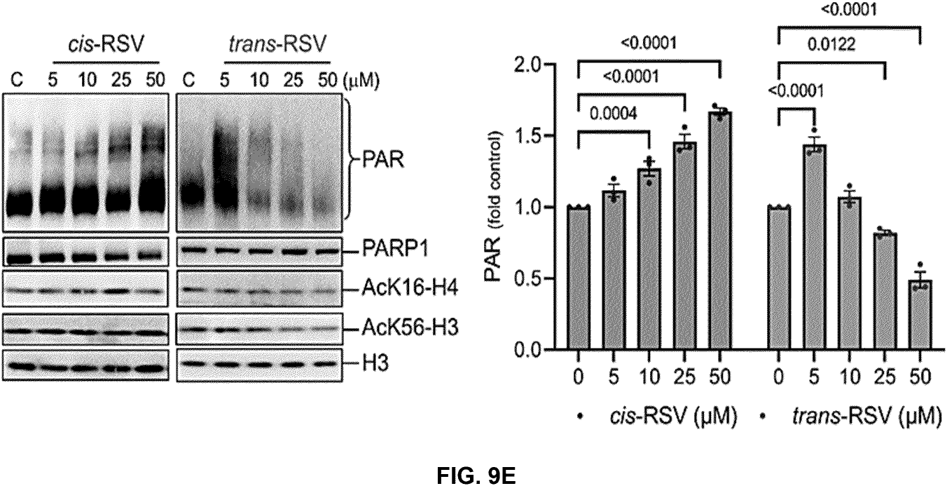

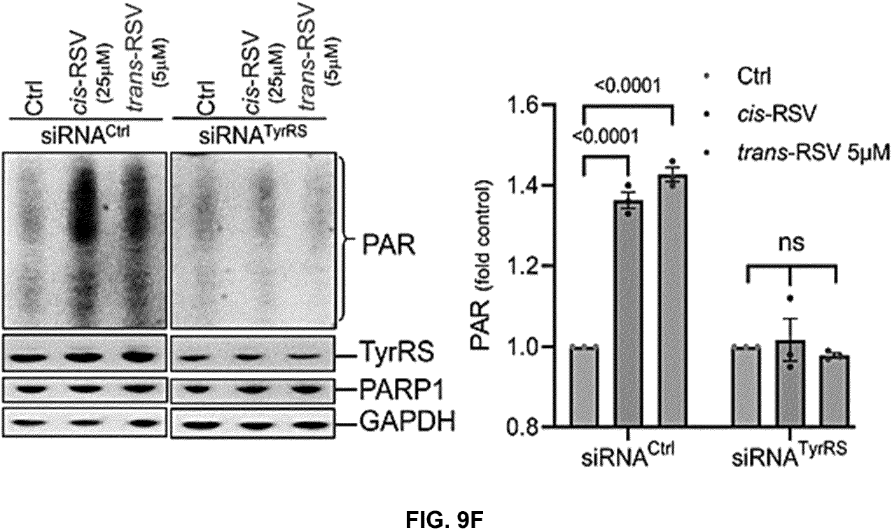

Protein synthesis is mainly regulated at the initiation and elongation steps. Ser51 phosphorylation of eukaryotic initiation factor 2 alpha (p-eIF2α) by multiple kinases and Thr56 phosphorylation of eukaryotic elongation factor 2 (p-eEF2) by eEF2 kinase (eEF2K) inhibit protein synthesis at the initiation and elongation steps, respectively. The protein kinase mammalian target of rapamycin (mTOR) inhibits eEF2K to activate protein synthesis (FIG. 9A). Although it is counterintuitive that elevated levels of tyrosine are inhibitory for TyrRS and protein synthesis in a p-eIF2αindependent manner, for which it is required, it was also noted that tyrosine co-instantaneously activates the assembly of eukaryotic initiation factor 4 F (eIF4F) and phosphorylation of ribosomal protein S6 kinase beta-1 (S6K1). More importantly, despite a significant decrease in the neurite levels of TyrRS, surprisingly, tyrosine did not induce neurite degeneration (FIG. 1A) and TyrRS levels were restored in 16-24 hr (FIG. 2A). These observations suggest that the effect of tyrosine on TyrRS levels is reversible and neurotrophic factors that activate protein synthesis may stimulate the de novo synthesis of TyrRS. The neurotransmitter dopamine (DA), which is decreased during aging, and in the affected brain regions of AD patients, is generated from tyrosine (L-Tyr → L-DOPA → DA). Interestingly, DA is known to activate eEF245, potentially by activating mTOR, resulting in the stimulation of neuronal protein synthesis and memory formation. Because BDNF, which is also depleted in AD brains, activates eEF2 and stimulates the de novo synthesis of neuronal TyrRS13 (FIG. 9B and FIG. 9C), we pondered if DA would also increase neuronal TyrRS levels. Consistent with our hypothesis, treatment with DA increased the protein levels of TyrRS (FIG. 2B), and the effects of DA were abrogated by rapamycin (Rapa) (FIG. 2C). Although tyrosine does not affect eIF2α function, it was observed that treatment with tyrosine inhibited eEF2 (FIG. 2D). These data suggest that beyond facilitating the degradation of TyrRS, tyrosine also inhibits the de novo synthesis of TyrRS, potentially at the elongation step through increased phosphorylation of eEF2, whereas BDNF and DA stimulate TyrRS synthesis (FIG. 2E).

### Cis-RSV and Trans-RSV Have Opposite Effects on TyrRS Levels in Neurons

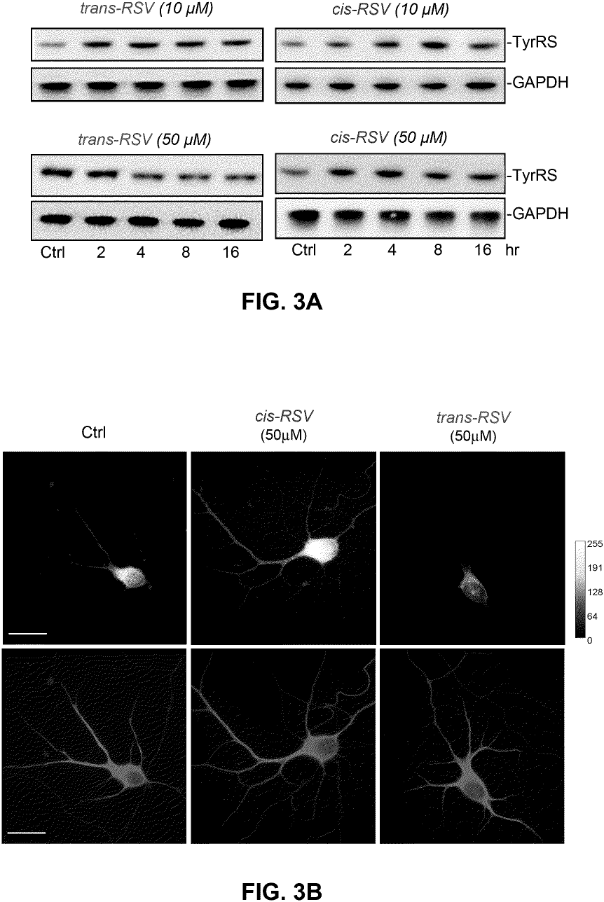

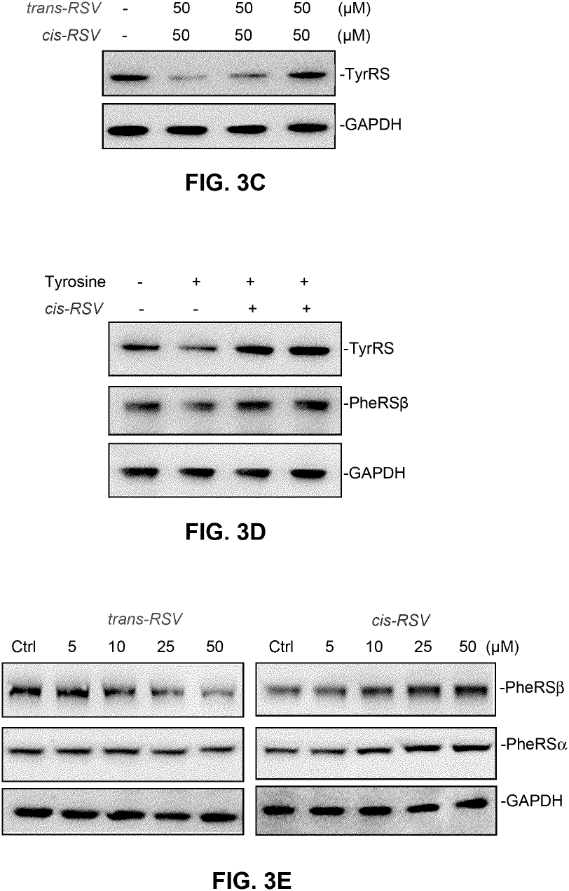

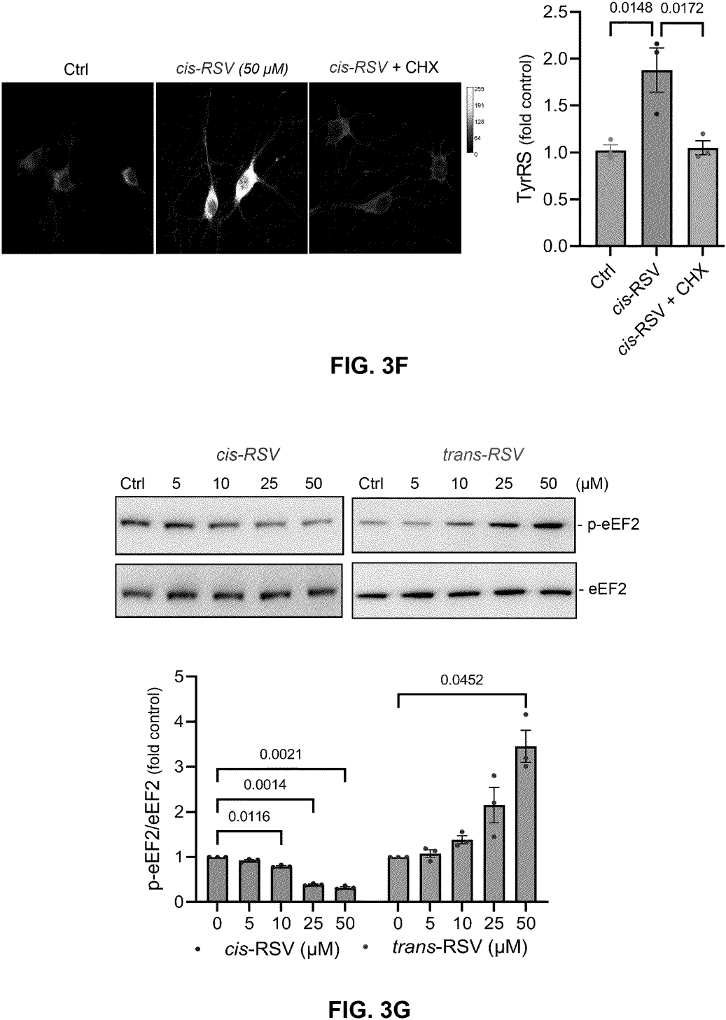

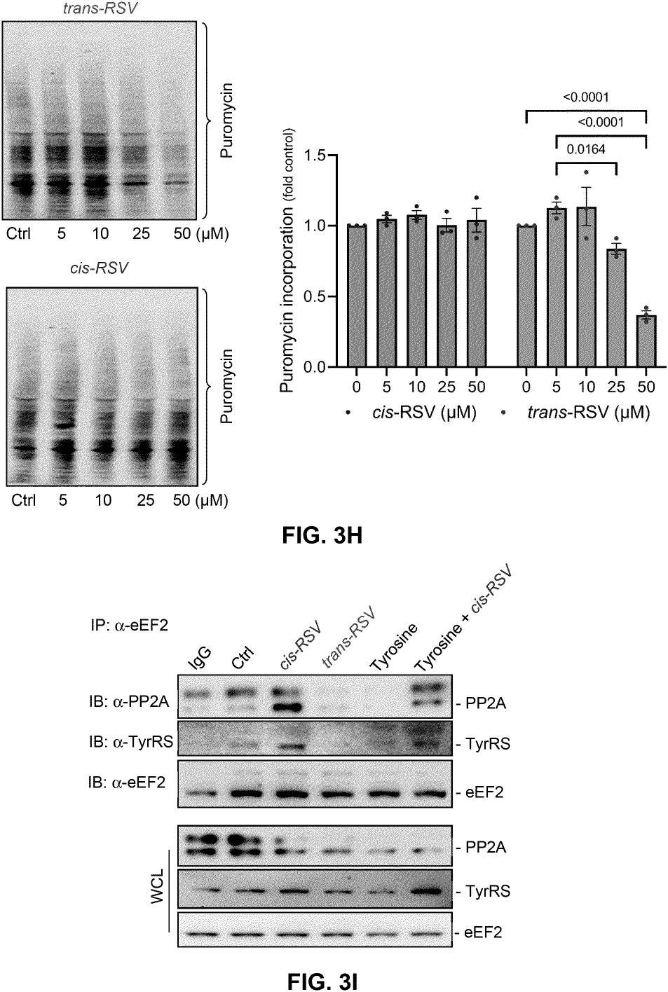

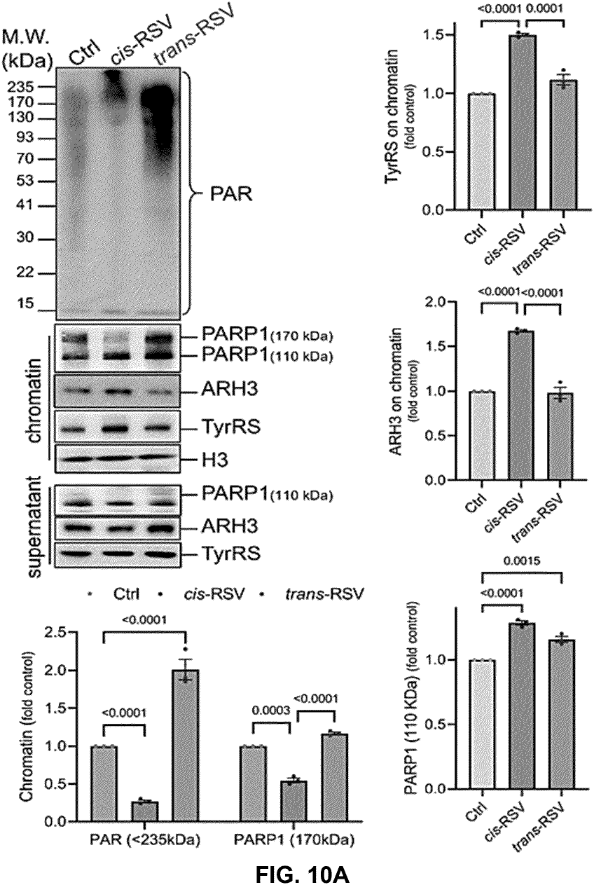

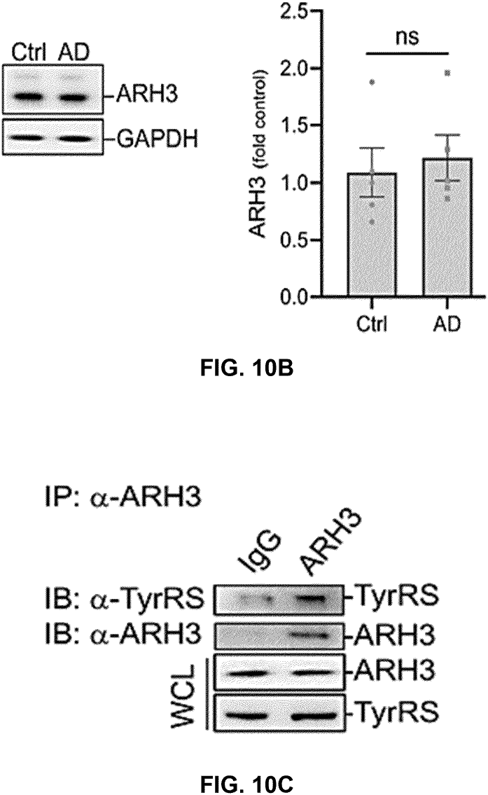

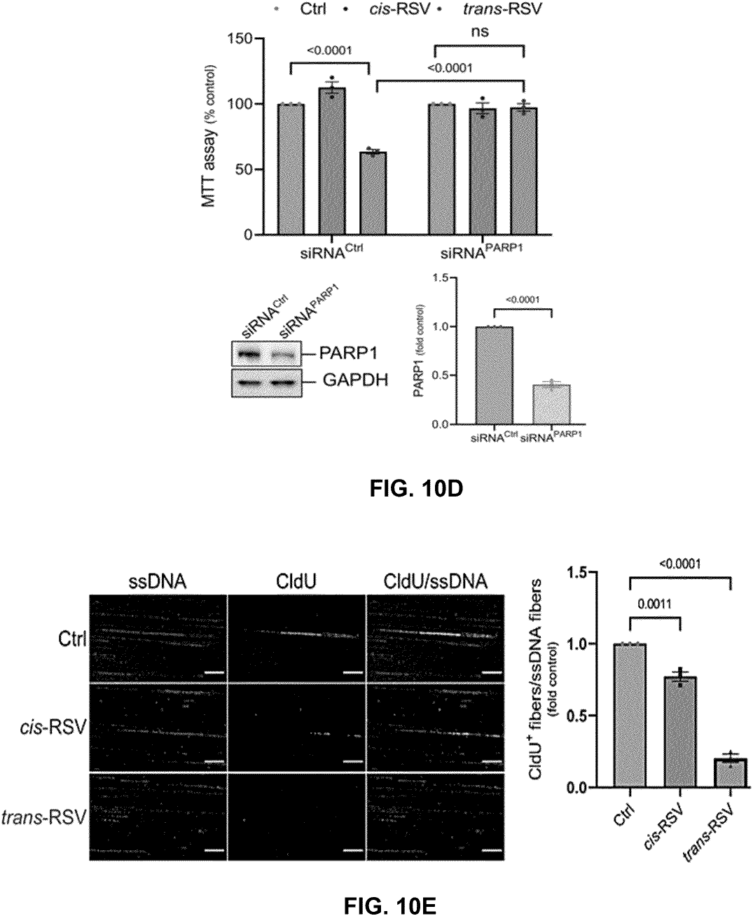

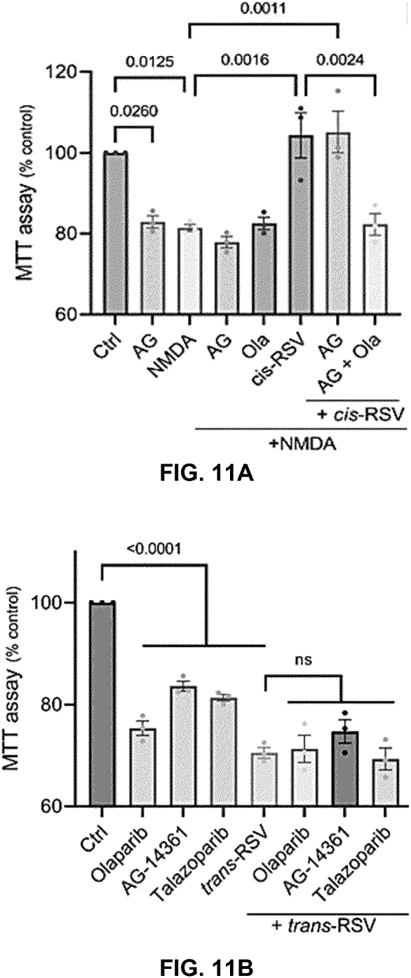

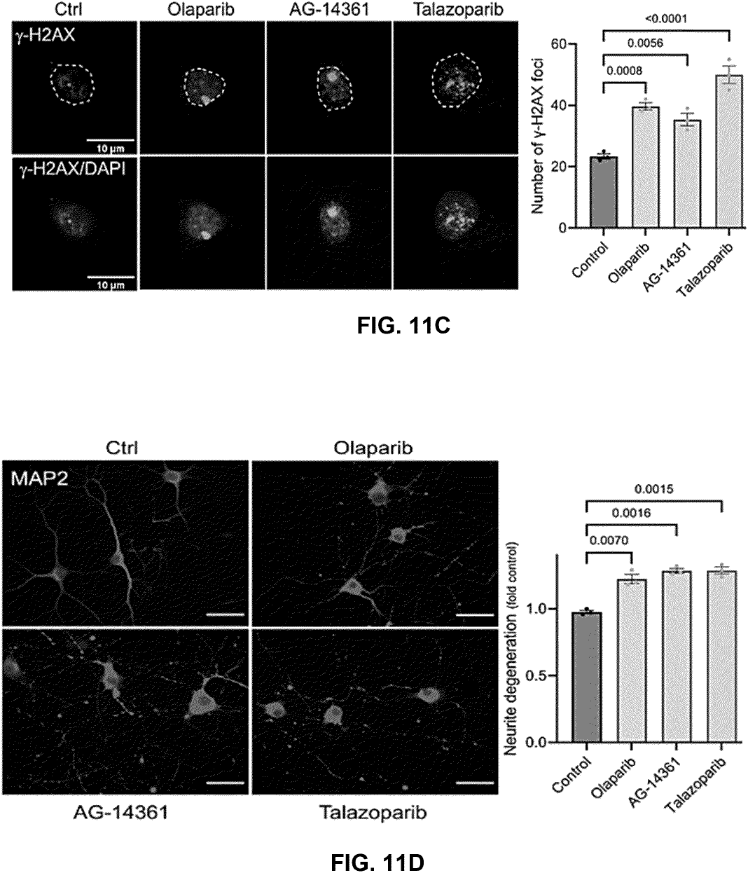

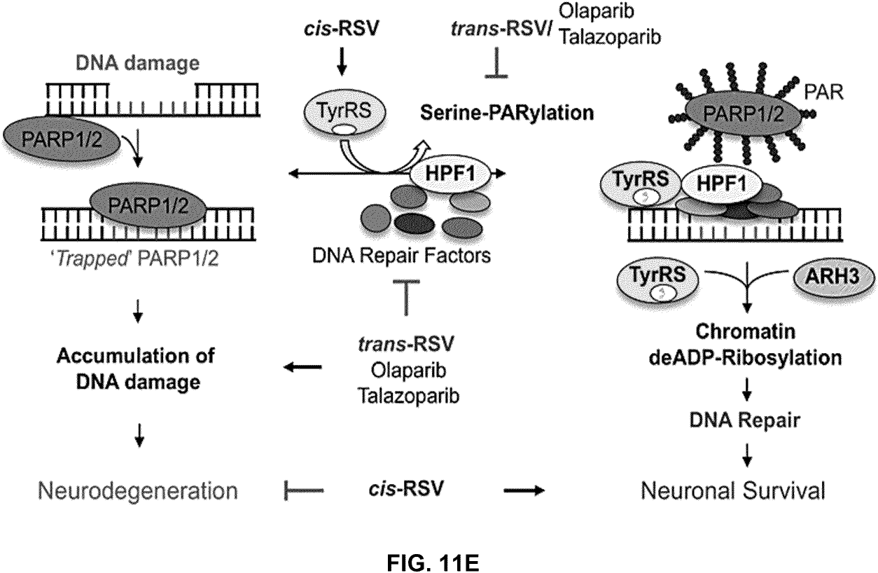

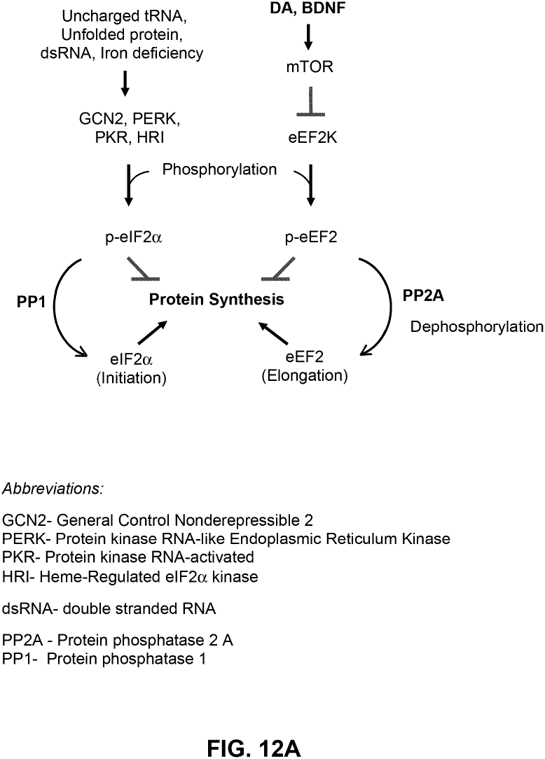

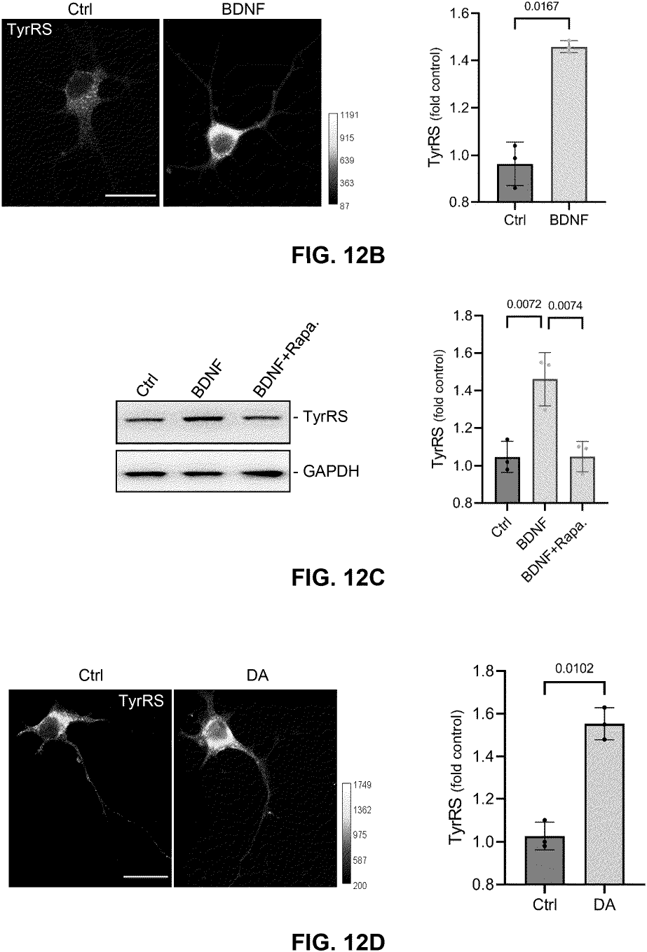

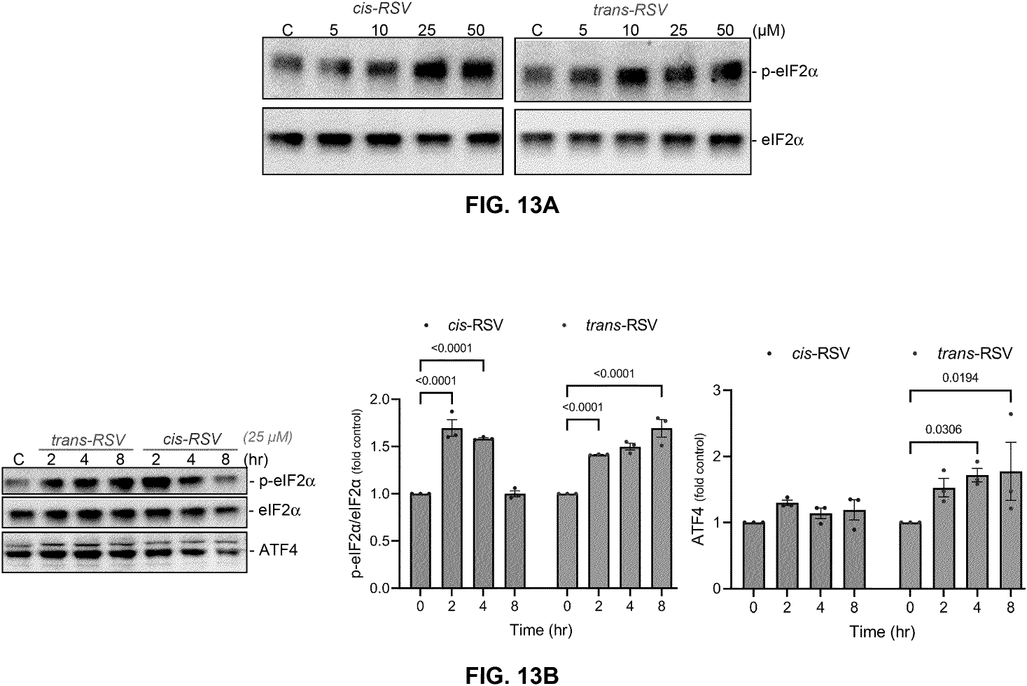

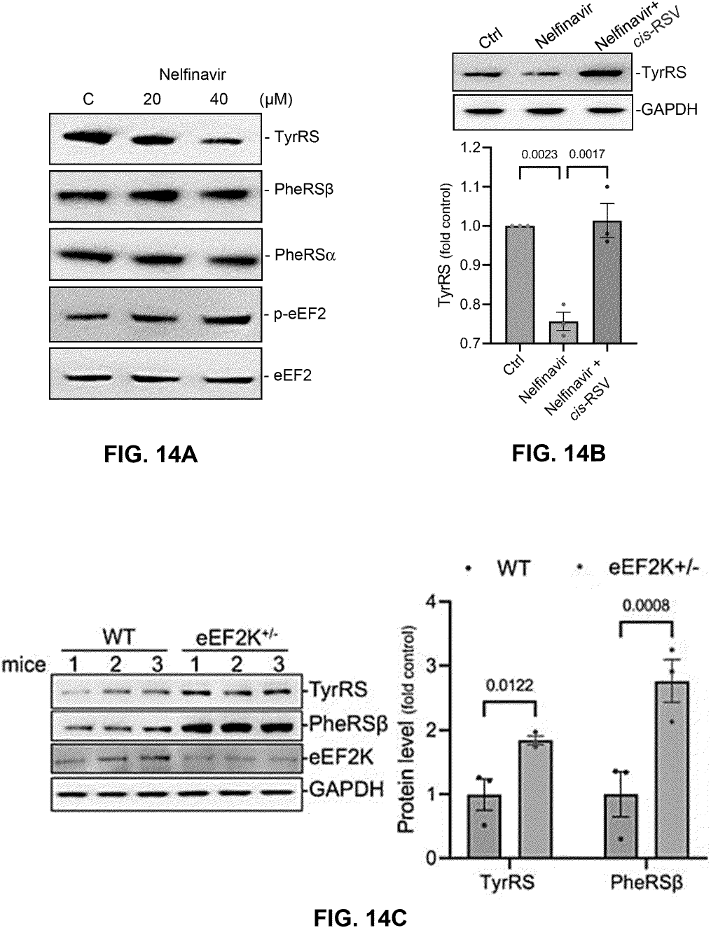

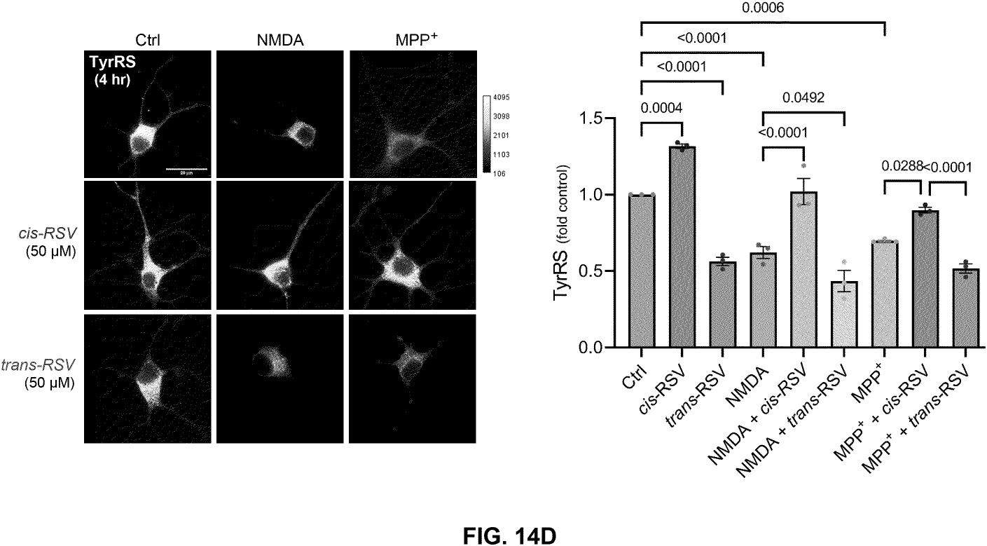

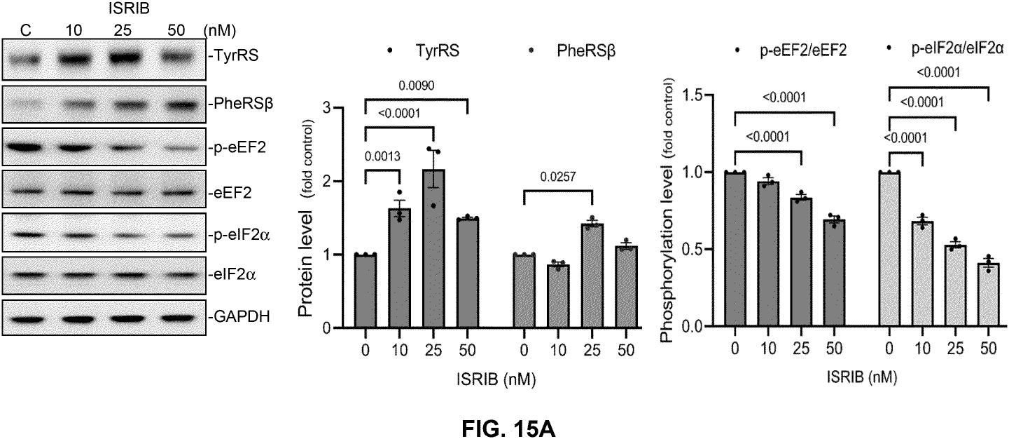

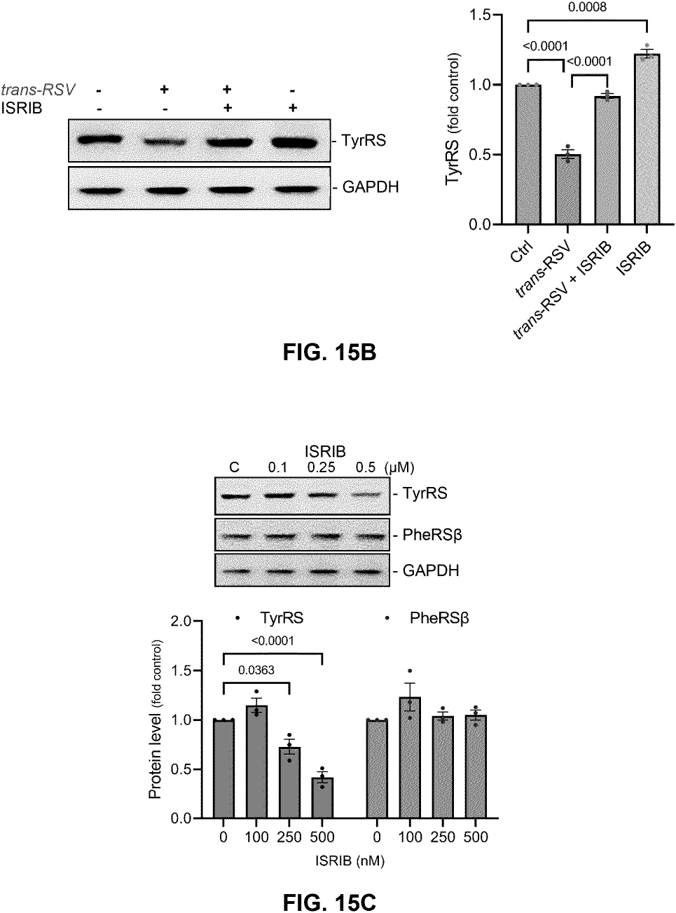

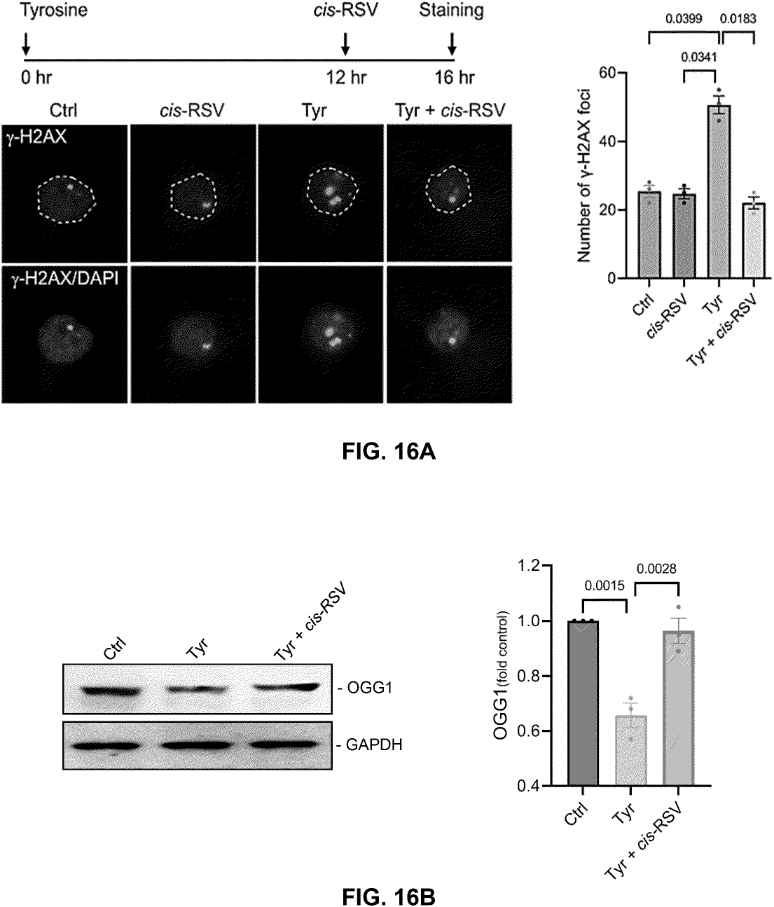

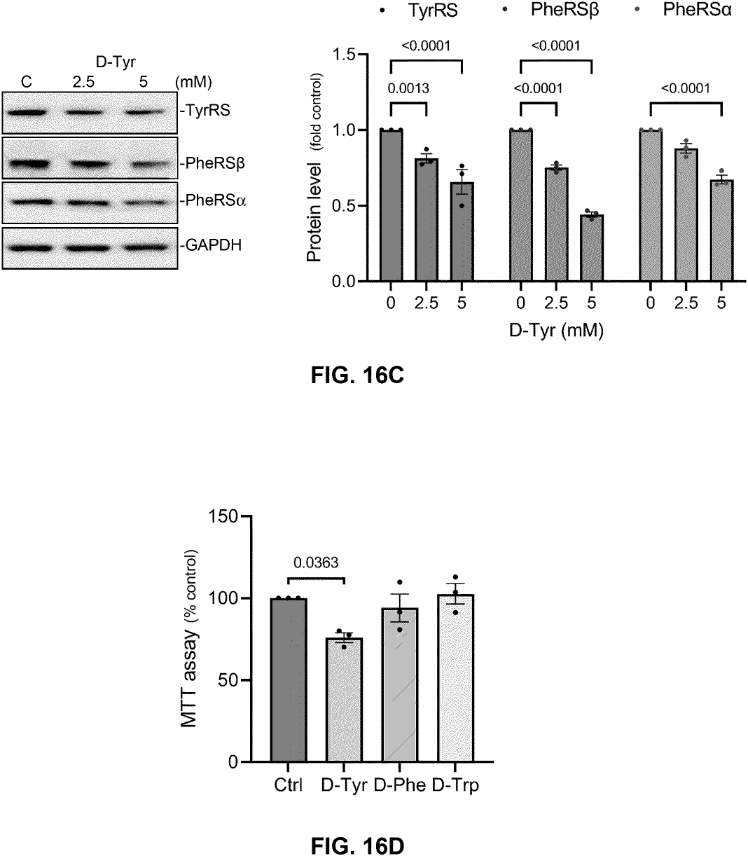

Natural RSV exists as a mixture of cis-RSV and trans-RSV and both are stable for at least six weeks at 4° C., and in the cell for 24 hr. However, the sulfate metabolites of trans-RSV mainly generate cis-RSV, suggesting that trans-RSV can be metabolically converted to cis-RSV in the cell. Clinical studies using 1,000 mg/dose trans-RSV (>99% pure) reported a peak trans-RSV plasma concentration of 137 µM and 50-640 µM trans-RSV accumulation in human tissues. Clinical studies using low-dose trans-RSV (50-75 mg/dose, e.g., 6-9 µM plasma concentration of trans-RSV/dose) reported CR-like benefits in postmenopausal women and in patients with heart failure. In contrast, clinical studies with 1,000 mg/dose trans-RSV resulted in brain volume loss in AD patients and increased CVD risk in older adults. However, it is noted that higher tyrosine level is a common biomarker for all the diseases/conditions mentioned above. Because the brain TyrRS levels correlate with cognitive performance in humans, it was hypothesized that cis-RSV and trans-RSV would exert differential effects on neuronal TyrRS levels and protein synthesis. Consistent with the results in clinical trials, a low dose of trans-RSV (10 µM) increased TyrRS and the high dose (50 µM) decreased it, whereas cis-RSV (10-50 µM) increased TyrRS protein levels both in the nucleus and neurites (FIG. 3A), mimicking the effects of reduced levels of tyrosine that increase TyrRS in the nucleus and neurites (FIG. 1B). cis-RSV rescued the effects of trans-RSV and tyrosine-mediated decrease in TyrRS levels in a dose-dependent manner (FIG. 3C). High concentration trans-RSV decreased PheRSβ, not PheRSα levels while low concentration cis-RSV increased their levels (FIG. 3E). Further, cis-RSV-mediated increase in TyrRS level was abrogated by CHX (FIG. 3F), suggesting that cis-RSV increases TyrRS in a protein synthesis-dependent manner. While cis-RSV triggered a transient increase in the levels of p-eIF2α (FIG. 10A and FIG. 10B), trans-RSV sustained the inhibition of eIF2α (FIG. 10A and FIG. 10B) and increased p-eEF2 levels (FIG. 3G). However, in contrast, cis-RSV activated the dephosphorylation of eEF2 (FIG. 3G). Finally, consistent with the dramatic depletion of TyrRS and increased levels of p-eIF2α and p-eEF2, treatment with trans-RSV inhibited global protein synthesis as measured by the puromycin incorporation assay (FIG. 3H). Because CR decreases serum tyrosine levels and decreased tyrosine levels increase TyrRS, taken together, these data indicate that cis-RSV may act as a potential CR mimetic by increasing TyrRS levels. Because eEF2 regulates the elongation phase of protein synthesis and eEF2K is the only known kinase that inhibits eEF2 (FIG. 9A), the present inventors wondered if direct pharmacological activation of eEF2K using nelfinavir would deplete TyrRS. Interestingly, nelfinavir depleted neuronal TyrRS, which was rescued by cis-RSV (FIG. 11A; FIG. 11B), suggesting that protein synthesis is required to maintain TyrRS levels under normal conditions. To determine the mechanism of cis-RSV-mediated activation of eEF2 through dephosphorylation, it was tested whether cis- and trans-RSV modulate the interaction of TyrRS with eEF2 and protein phosphatase 2 (PP2A) (FIG. 9A). It was observed that cis-RSV facilitated the interaction of eEF2 with TyrRS and PP2A, whereas trans-RSV and tyrosine decreased their interactions (FIG. 3I). These data suggest that cis-RSV and tyrosine modulate TyrRS levels at the elongation step and cis-RSV mimics DA and BDNF in activating the de novo synthesis of TyrRS. Furthermore, similar to 6-OHDA (FIG. 8C), other neurotoxic agents such as the N-Methyl-D-aspartate (NMDA) and the mitochondrial toxin 1-methyl-4-phenylpyridinium (MPP+) also decreased the levels of neuronal TyrRS after 4 hr of treatment; cis-RSV suppressed this effect, whereas trans-RSV exacerbated it, suggesting that neuronal TyrRS is a potential target of multiple neurotoxic agents.

### Pharmacological Activation of Protein Synthesis Increases TyrRS Levels in Neurons

Regulation of eIF2α modulates motor and cognitive function, and neuronal survival. Pharmacological activation of protein synthesis using integrated stress response inhibitor (ISRIB) protects against age-related memory deficits. Treatment with ISRIB (5-50 nM) consistently increased the protein levels of TyrRS and PheRSβ (FIG. 11A) and rescued trans-RSV mediated depletion of TyrRS (FIG. 11B). Interestingly, ISRIB stimulated the dephosphorylation of eEF2, potentially mediated through TyrRS. However, higher doses of ISRIB (250-500 nM) decreased TyrRS levels. Interestingly, TyrRS (but not PheRSβ) is among the activating transcription factor 4 (ATF4) target genes that are specifically upregulated in integrated stress response (ISR). Because there is no indication that the regulation of gene expression in ISR is different in neurons and ISRIB inhibits ATF4 target gene expression, it is highly likely that the concentration dependent decrease in TyrRS level is due to the repression of TyrRS upon higher doses of ISRIB. Because tyrosine decreased neurite TyrRS levels and sleep stimulated synaptic protein synthesis, we wondered if the synaptic TyrRS is circadian regulated. Re-analysis of the mouse circadian proteomic and metabolomic data showed that synaptic protein level of only TyrRS (among all the aaRSs) is circadian-regulated and is inversely correlated with tyrosine levels. Further, re-analysis of the human metabolome showed that sleep deprivation increases serum tyrosine levels. Collectively, these data suggest that tyrosine is a potential endogenous modulator of the synaptic and nuclear TyrRS.

### Tyrosine Induces Oxidative DNA Damage in Neurons and Cis-RSV Protects Against It

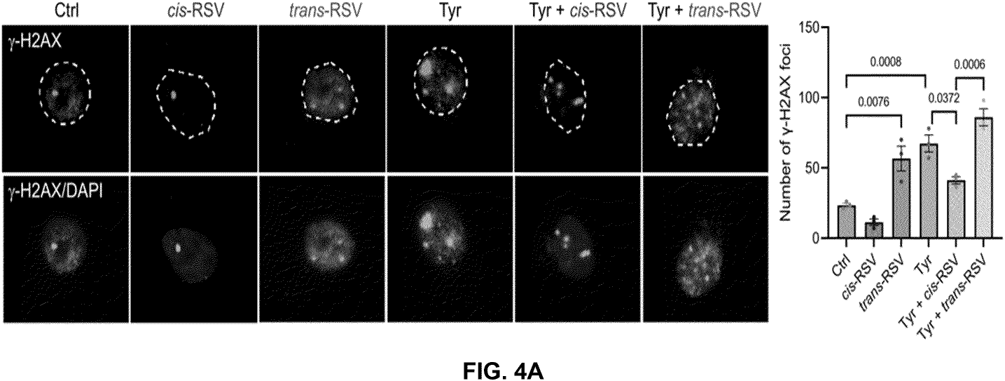

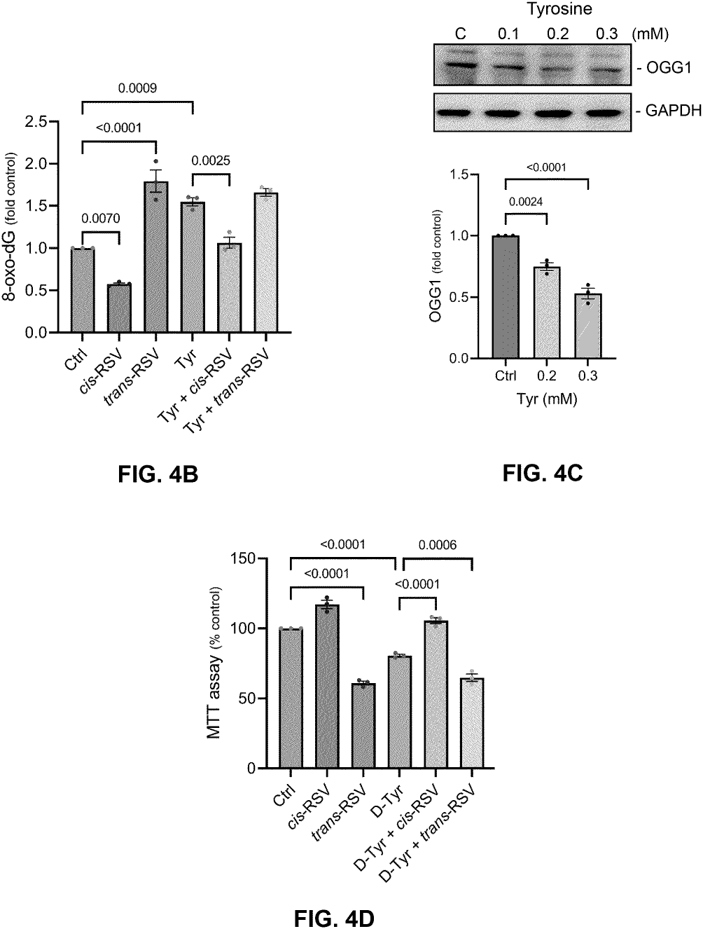

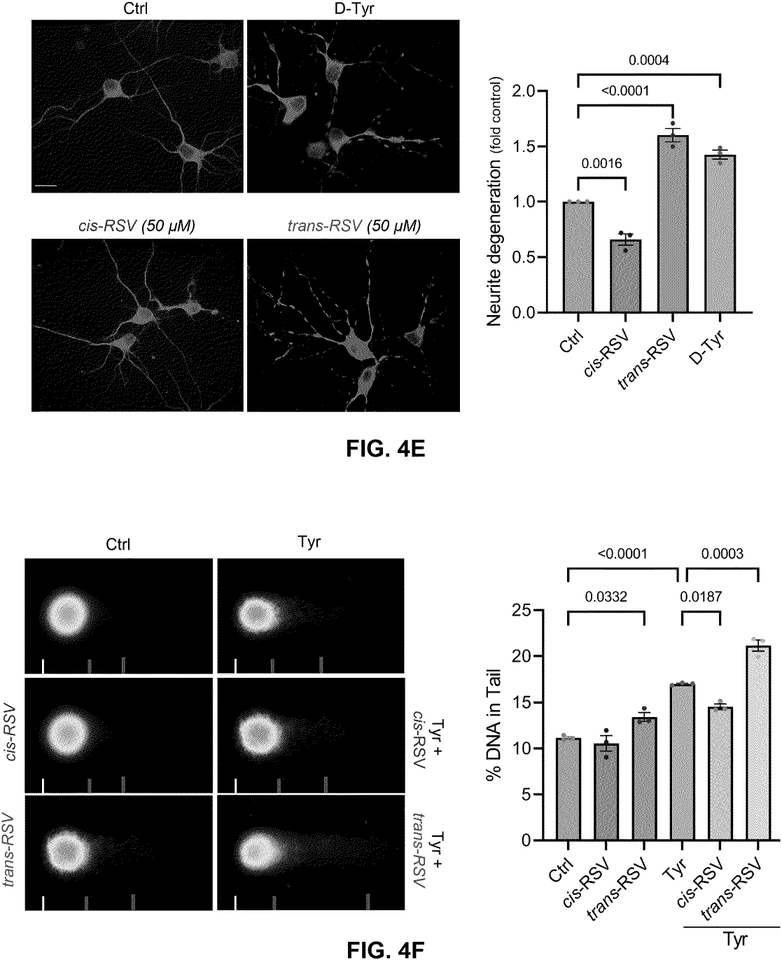

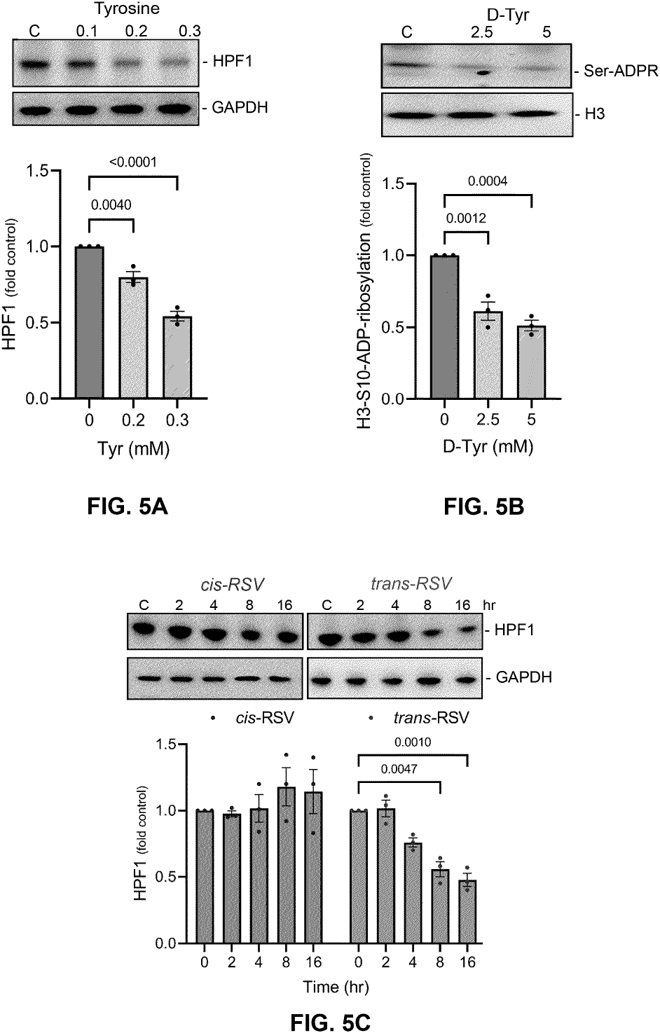

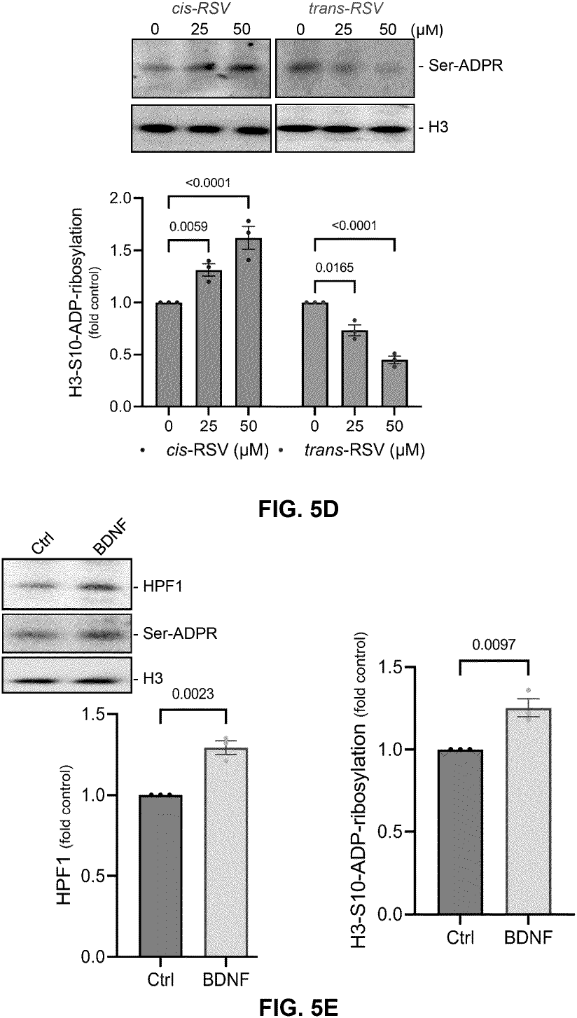

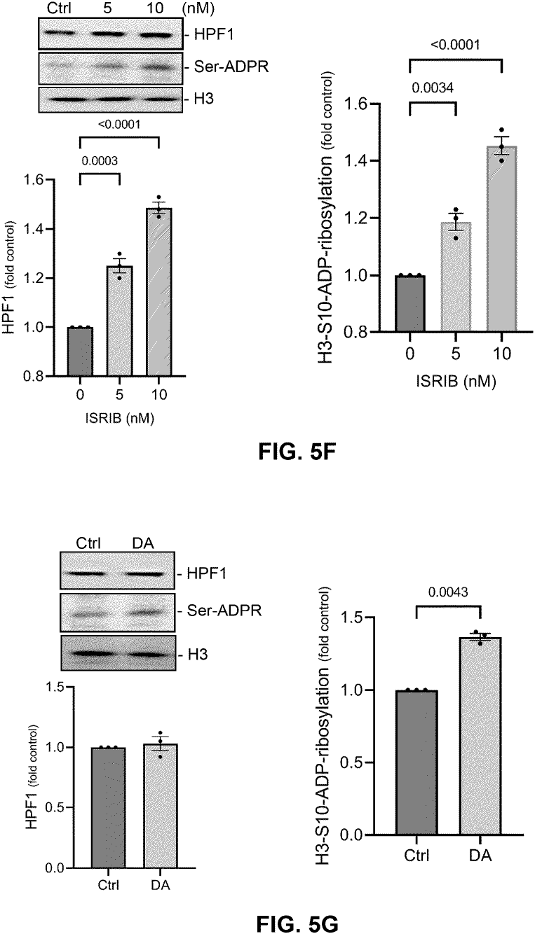

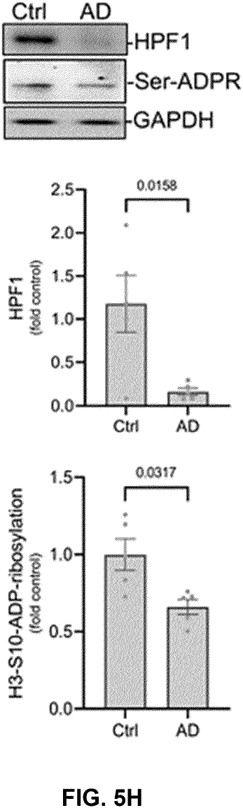

Human aging and neurodegenerative diseases accumulate oxidative DNA damage-associated mutations in neurons whereas CR protects against oxidative stress. While sleep deprivation causes oxidative DNA damage, sleep stimulates neuronal DNA repair through unknown mechanisms. Because TyrRS shows moonlighting functions in DNA repair signaling, it was hypothesized that decreased tyrosine levels at night may switch the function of a fraction of TyrRS from protein synthesis to DNA repair, and conversely, aging and neurodegenerative diseases that increase tyrosine levels may inhibit TyrRS-mediated DNA repair. Consistent with our hypothesis, treatment with tyrosine resulted in the accumulation of γ-H2AX foci (a marker of DNA damage) and 8-oxo-2′-deoxyguanosine (8-oxo-dG, a marker of oxidative DNA damage). These observations indicate that compounds that mimic ‘tyrosine-like’ conformation in TyrRS may induce oxidative DNA damage. Conversely, compounds that mimic a ‘tyrosine-free’ conformation in TyrRS may shift its function to facilitate neuronal DNA repair. To test this possibility, we treated neuronal cultures with cis-RSV, which evokes a ‘tyrosine-free’ conformation, and trans-RSV, which mimics a ‘tyrosine-like’ conformation in TyrRS28. While cis-RSV rescued tyrosine-induced accumulation of γ-H2AX and 8-oxo-dG, trans-RSV itself caused the accumulation of γ-H2AX and 8-oxo-dG. Interestingly, cis-RSV reduced γ-H2AX levels in neuronal cultures even after 12 hr of tyrosine pretreatment had already caused substantial amount of DNA damage suggesting that beyond prevention, cis-RSV may reverse existing neuronal DNA damage as well. Further, tyrosine decreased 8-oxoguanine-DNA glycosylase (OGG1) levels, which was rescued by cis-RSV. 8-oxo-dG is highly mutagenic, driving a G▪C → T▪A transversion. Consistently, mutagenic frequency increases during aging and γ-H2AX and 8-oxo-dG are accumulated in aged and AD neurons. Further, D-Tyr, which does not get converted to DA but is activated by TyrRS2, induced neurotoxicity, whereas D-Phe and D-Trp had no significant effects. cis-RSV protected against D-Tyr mediated neurotoxicity but not trans-RSV (FIG. 4E). To gain direct evidence for DNA damage, we also conducted the comet assay. Cortical neurons were treated with trans-RSV and L-tyrosine either alone or in combination and found that both trans-RSV, L-tyrosine, and their combination significantly increased DNA damage as measured by the increase in percentage (%) of DNA in the comet tail. In contrast, cis-RSV treatment did not cause an increase in percentage of DNA in the comet tail and attenuated the increase in DNA damage caused by Tyr (FIG. 4F). Since CR activates BER, this data indicates that cis-RSV may act as a CR mimetic by activating BER.

### Histone Serine-ADP-ribosylation Is Decreased in AD Brains and BDNF and Dopamine Increase Its Levels.

Histone poly-ADP-ribosylation factor (HPF1)-dependent serine-ADP-ribosylation is essential for PARP1-dependent DNA repair and histone H3 is one of the best characterized targets of HPF1/PARP1-mediated serine-ADP-ribosylation. Because AD brains show accumulation of neuronal DNA damage, the levels of HPF1′s and histone serine-ADP-ribosylation’s ability to affect AD brains was investigated. Our analysis showed reduced HPF1 levels, as well as decreased histone H3 serine-ADP-ribosylation, in the hippocampal tissues of AD patients. Because tyrosine inhibits protein synthesis, it was determined whether treatment with tyrosine and trans-RSV would modulate HPF1 at the translational level. Similarly, we wondered if treatment with DA, BDNF or ISRIB would affect the protein levels of HPF1. It was observed that while treatment with cis-RSV stimulated serine-ADP-ribosylation, treatment with tyrosine and trans-RSV decreased HPF1 levels in addition to inhibiting histone H3 serine-ADP-ribosylation. Furthermore, treatment with either BDNF or ISRIB increased HPF1 levels along with the induction of histone H3 serine-ADP-ribosylation. Similarly, DA also stimulated histone H3 serine-ADP-ribosylation. Taken together, beyond DNA repair, these data indicate a potential role of serine-ADP-ribosylation in cognition and memory and provide a potential molecular basis for the reduced HPF1 levels and serine-ADP-ribosylation in the hippocampal tissues of AD patients.

### Cis-RSV Is Neuroprotective and Trans-RSV Is Neurotoxic

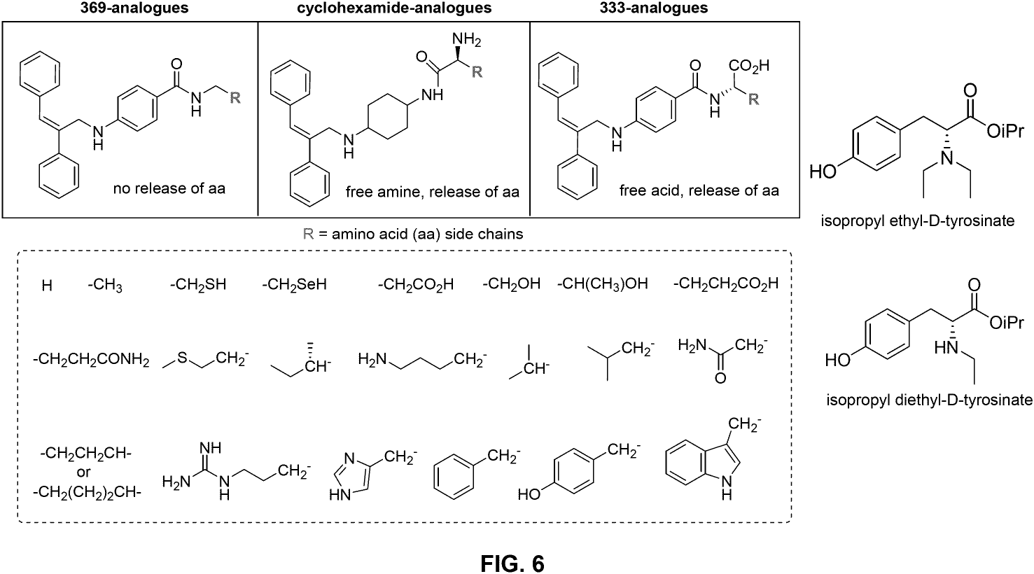

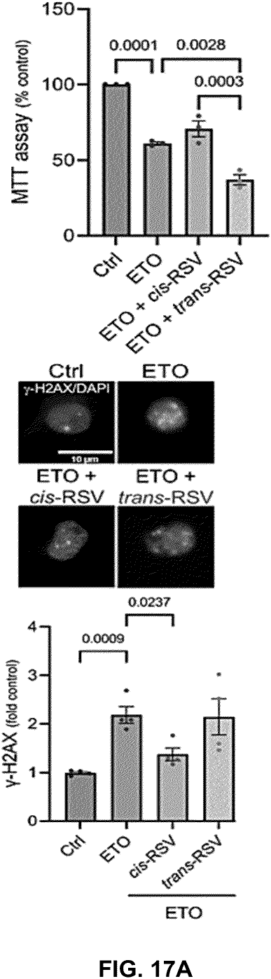

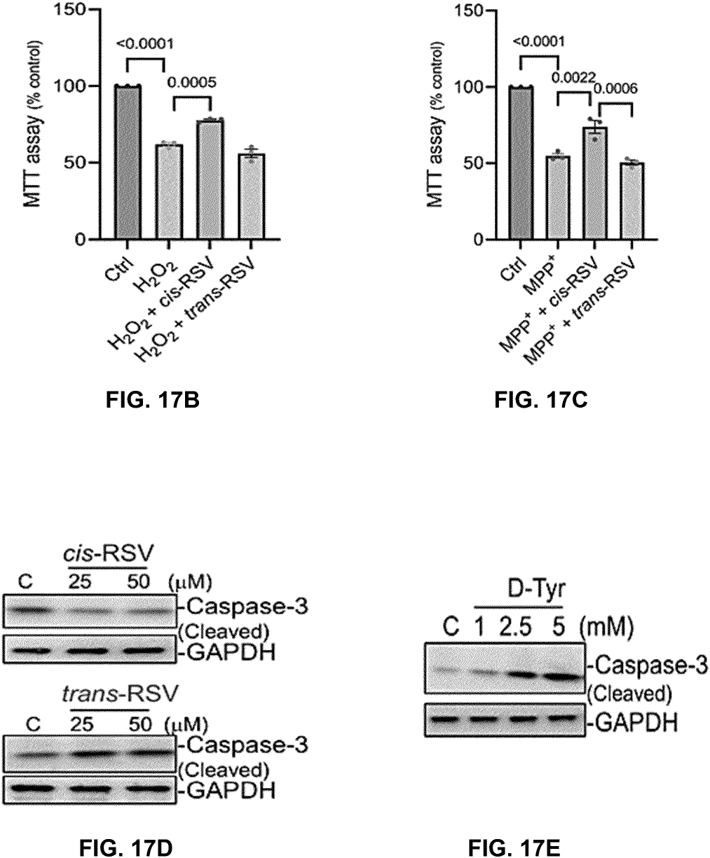

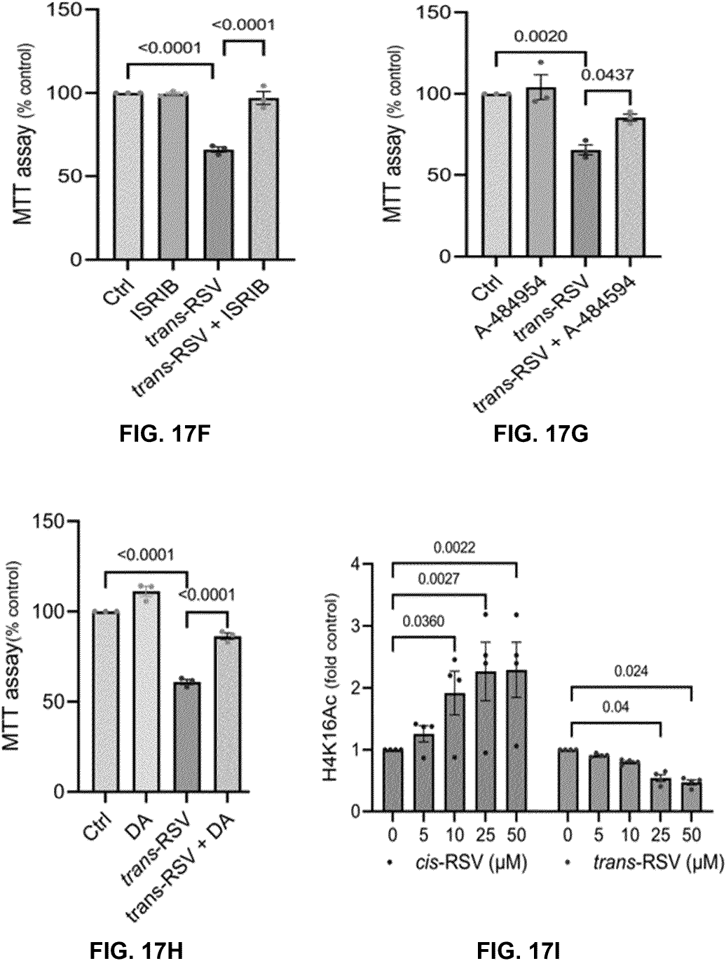

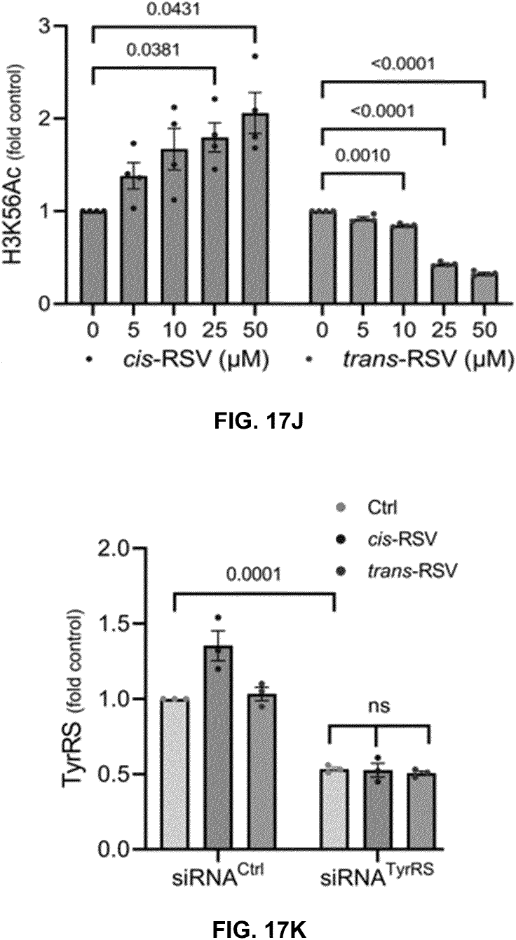

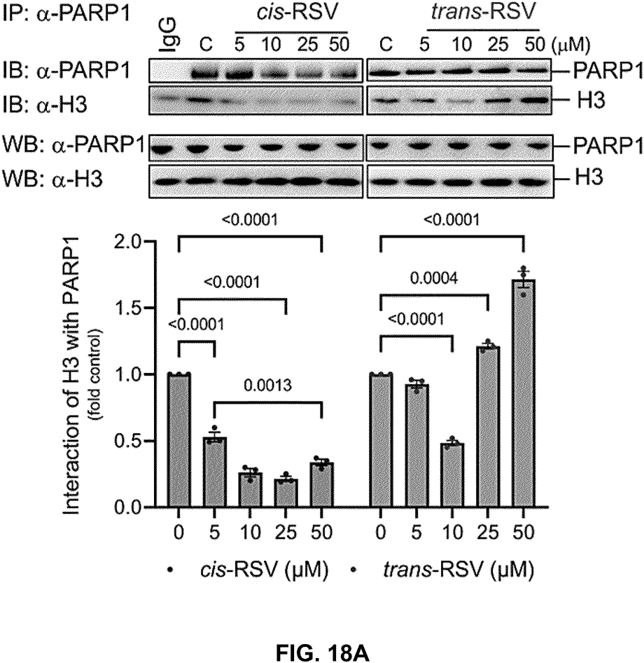

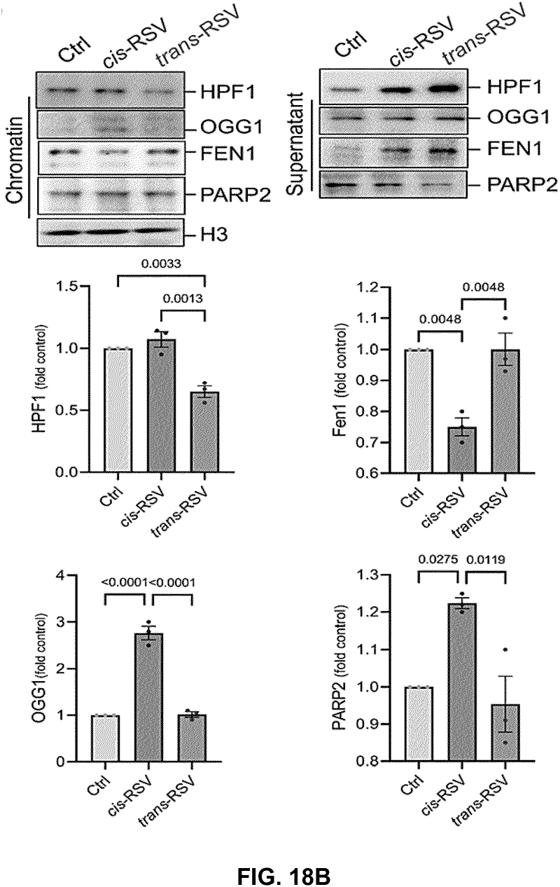

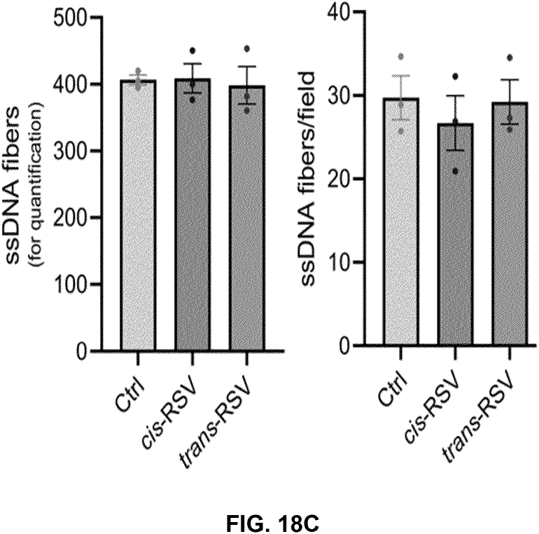

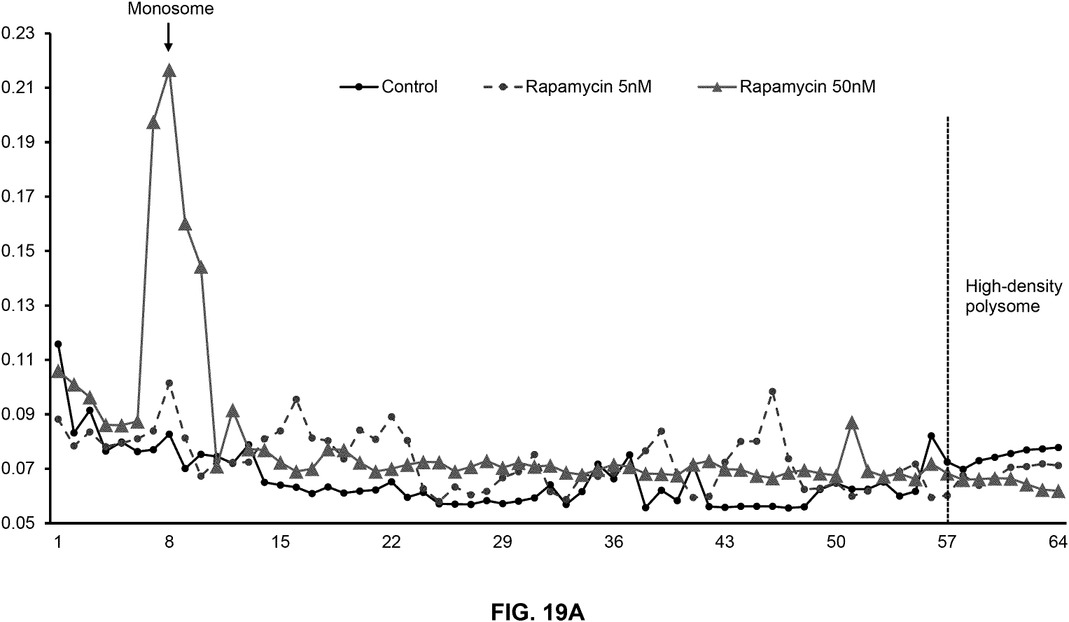

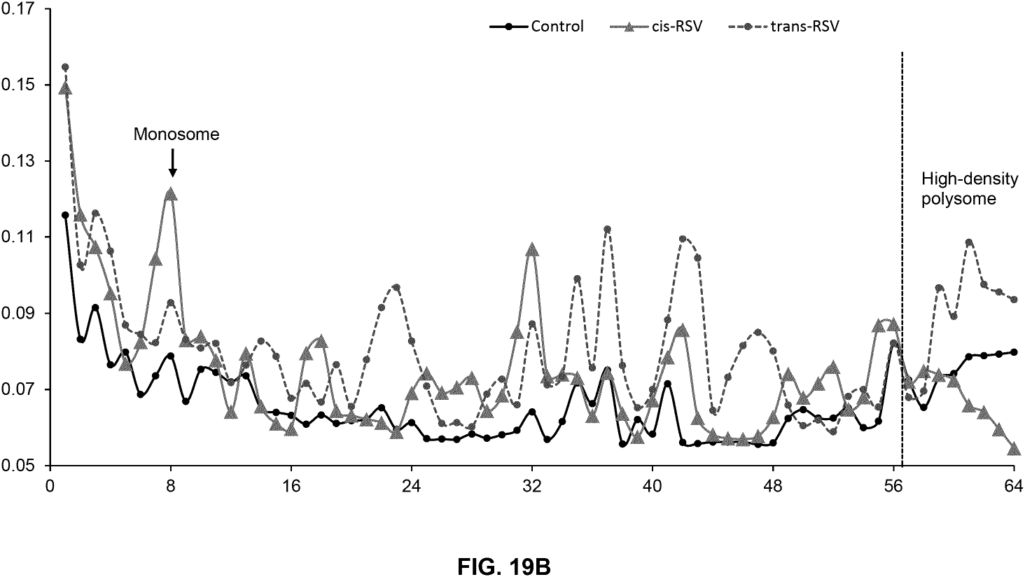

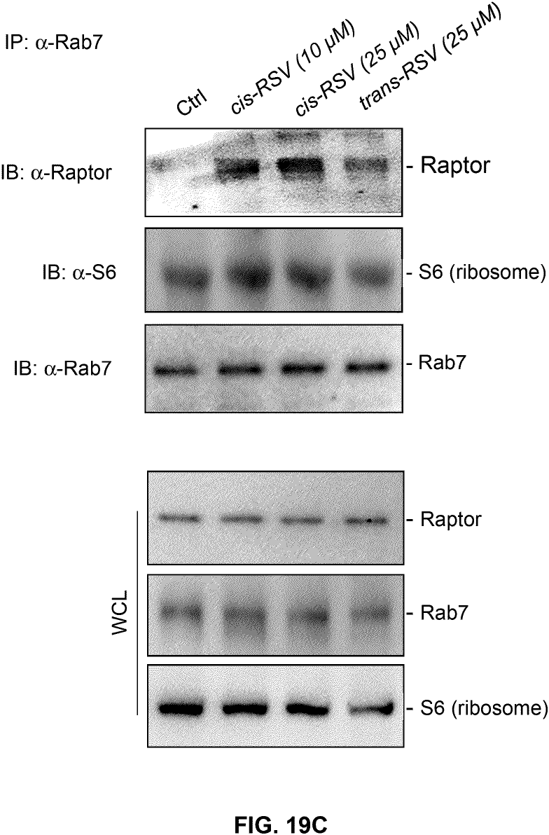

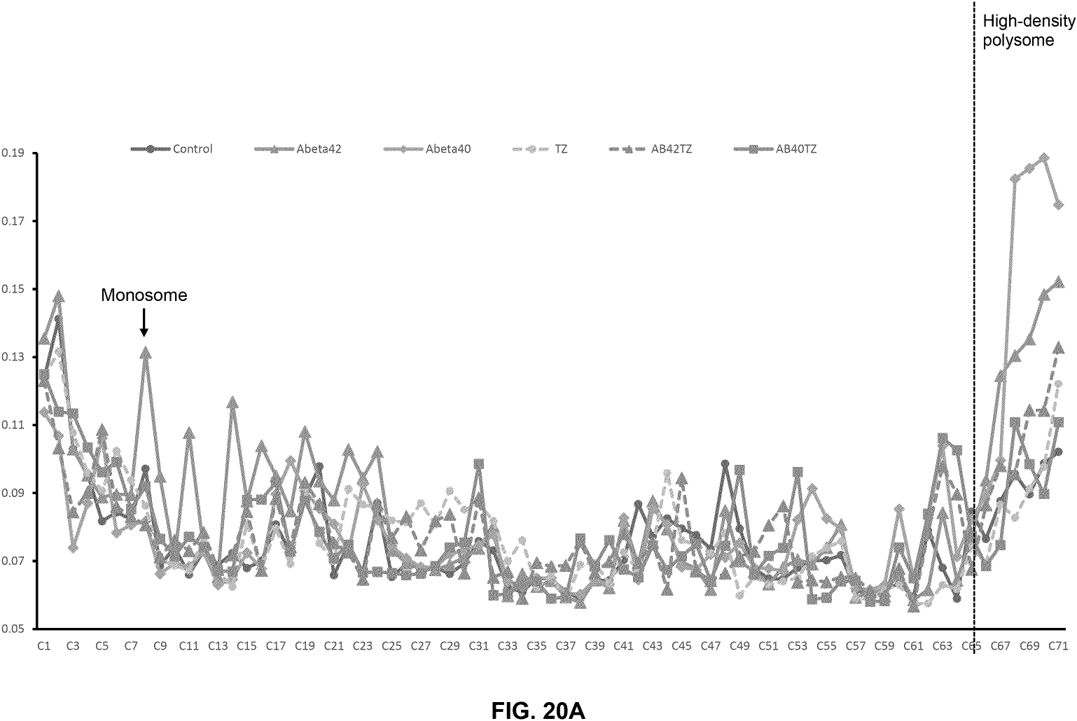

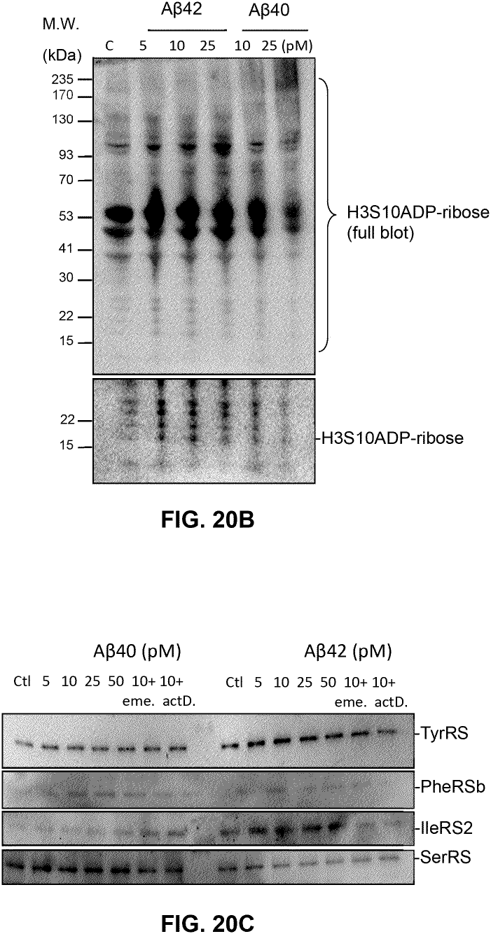

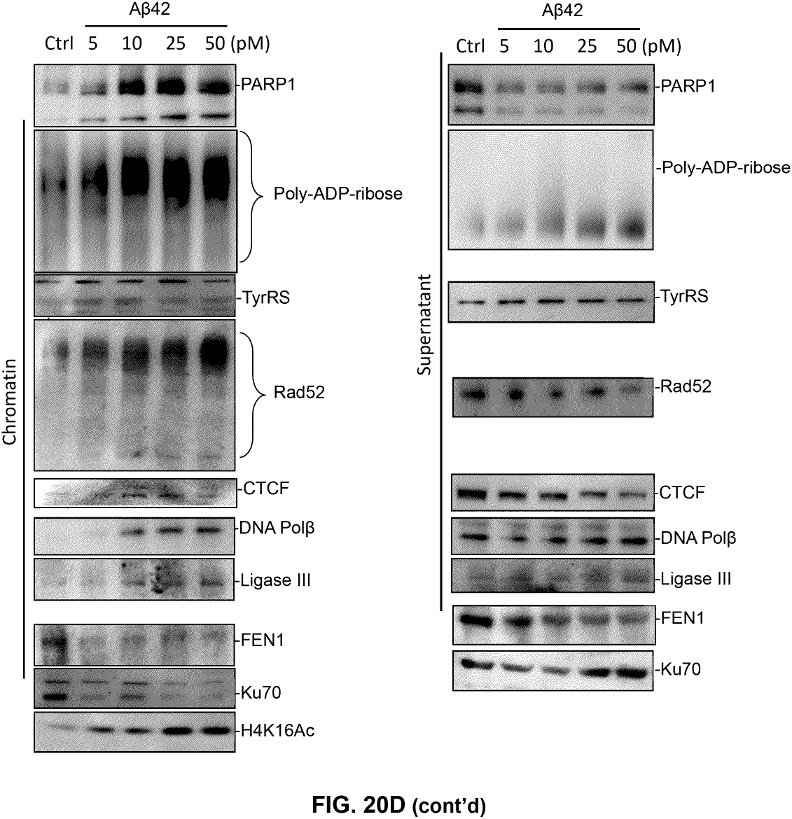

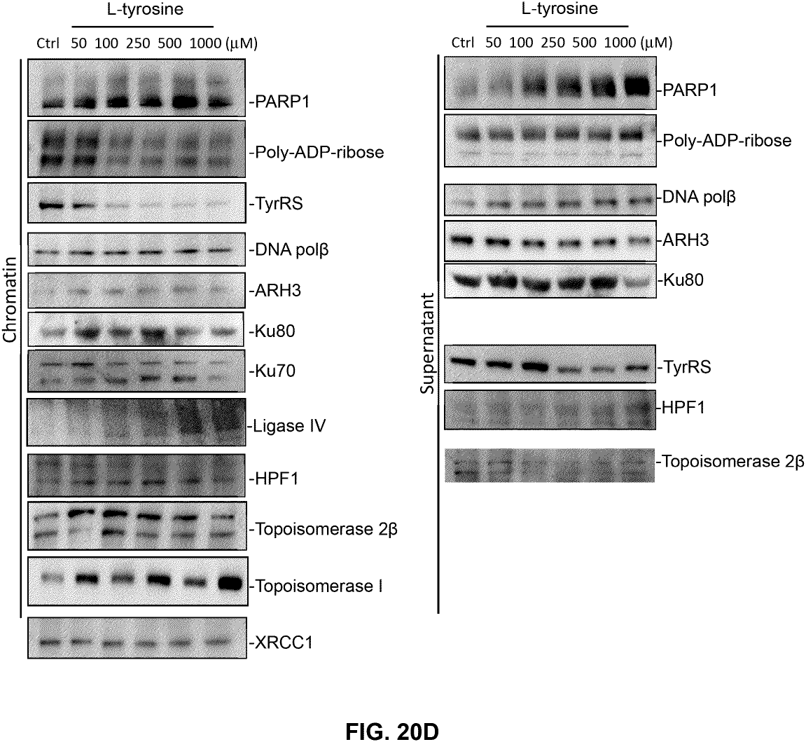

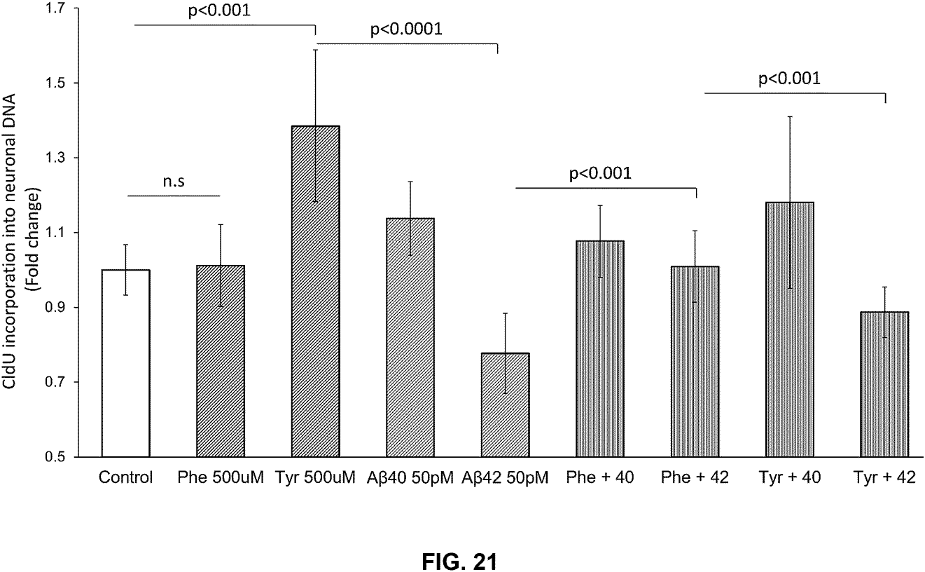

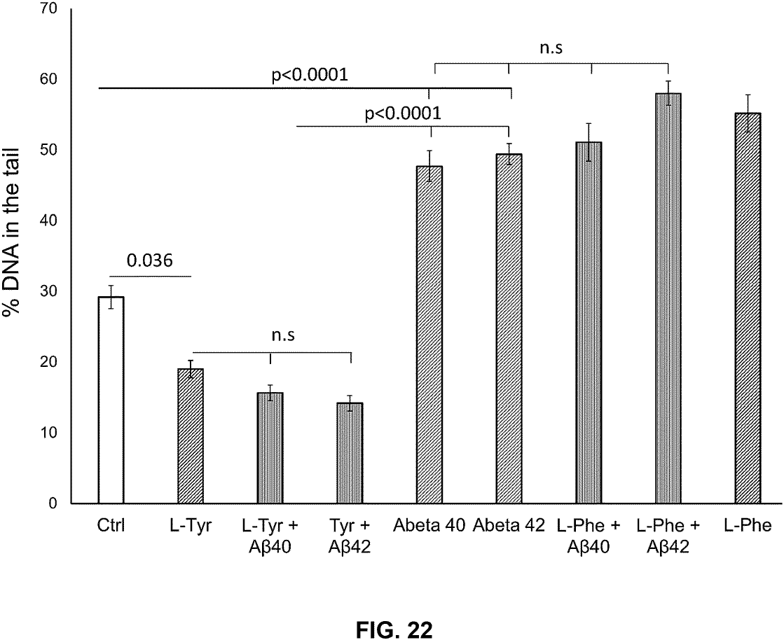

After showing that cis- and trans-RSV have opposite effects on neuronal oxidative DNA damage, we hypothesized that they would exert differential effects on neuronal survival-under-stress conditions. The effects of cis- and trans-RSV were analyzed on the survival of rat primary cortical neurons exposed to different stress agents to test this hypothesis. The effect of trans-RSV on NMDA mediated neurotoxicity showed a concentration-dependent dual response, where low concentrations of trans-RSV (≤ 10 mM) evoked protective effects, but the higher concentrations (≥ 25 mM) exacerbated the toxicity (FIG. 9A). In contrast, cis-RSV protected against NMDA-mediated toxicity in a concentration-dependent manner (FIG. 9B). Hence, the concentration-dependent dual response of trans-RSV on neuroprotection is consistent with trans to cis conversion at low concentrations, increasing TyrRS levels (FIG. 3A), and the retention of ‘trans-/tyrosine-like’ conformation at high concentrations, causing TyrRS depletion (FIG. 3A). Interestingly, cis-RSV (50 mM) suppressed the neurotoxicity induced by a DNA-damaging agent (etoposide, ETO) (FIG. 17A), oxidative stress (H₂O₂) (FIG. 17B), and mitochondrial inhibition (MPP⁺) (FIG. 17C), but trans-RSV (50 mM) did not protect against these neurotoxic agents. Further, it was observed that cis-RSV-mediated rescue of ETO-mediated DNA damage is reflected by decreased levels of y-H2AX. To test if the observed effects of cis- and trans-RSV on neurotoxicity are mediated via TyrRS, siRNA knockdown of TyrRS (siRNA^{TyrRS}) in rat cortical neurons was carried out. Although TyrRS knockdown did not significantly affect the viability, it blunted the neuroprotective effects of cis-RSV and did not diminish the toxicity of trans-RSV (50 mM) upon NMDA treatment (FIG. 6C). These results suggest that the neuroprotective effect of cis-RSV (and low-dose trans-RSV) is TyrRS dependent, but the neurotoxic effect of trans-RSV is TyrRS independent. Moreover, it was found that trans-RSV (50 mM) by itself was neurotoxic in the rat primary cortical neuron cultures, whereas cis-RSV protected against the neurotoxicity induced by trans-RSV in a dose-dependent manner. Furthermore, a high concentration of trans-RSV and D-tyrosine increased the levels of cleaved caspase-3 (a marker of apoptosis) in rat primary cortical neurons. In contrast, cis-RSV decreased the levels of caspase-3 cleavage. However, treatment with either ISRIB, eEF2K inhibitor, or DA protected against neurotoxic effects of trans-RSV, indicating a critical role of sustained protein synthesis in neuronal DNA repair and survival.

### Cis-RSV and Trans-RSV Have Opposite Effects on the Auto-PARylation of PARP1

Similar to the circadian regulation of tyrosine, the auto-PARylation of PARP1 is also circadian-regulated. Previously, it was observed that tyrosine inhibits the auto-PARylation of PARP1, while cis-RSV induces a ‘tyrosine-free’ conformation in TyrRS to stimulate the auto-PARylation. Because trans-RSV mimics the ‘tyrosine-like’ conformation in TyrRS, the effects of cis- and trans-RSV on the auto-PARylation of PARP1 was analyzed. cis-RSV, converted from a low concentration of trans-RSV (5 mM) in solution, stimulates the auto-PARylation of PARP1, whereas higher concentrations of trans-RSV (≥ 25 mM) inhibit the auto-PARylation of PARP1. Interestingly, the apparent Ki value of trans-RSV-mediated inhibition of tyrosine activation by TyrRS in an ATP-PPi exchange assay was ~25 mM, which is the lowest concentration of trans-RSV that significantly inhibits the auto-PARylation of PARP1. In addition, it was previously shown that tyrosine inhibits auto-PARylation-dependent acetylation of proteins. Further, 500 mg/day dosing of trans-RSV (68.5 mM plasma level) inhibited the acetylation of H3 at lysine 56 (AcK56-H3) in humans. Consistently, trans-RSV (≥ 25 mM) inhibited H3 and H4 acetylation at lysines 56 and 16, respectively (AcK56-H3 and AcK16-H4), whereas cis-RSV increased the acetylation of H4 lysine 16 (AcK16-H4). Furthermore, TyrRS knockdown diminished the effects of low-concentration (5 mM) trans-RSV and cis-RSV (25 mM) on the auto-PARylation of PARP1, supporting our previous findings using low-concentration (≤ 5 mM) trans-RSV.

### Cis-RSV Stimulates the De-ADP-Ribosylation of Neuronal Chromatin

Although HPF1 and serine-ADP-ribosylation are decreased in AD brains, PARP1 can also PARylate on the glutamic/aspartic acid (Glu/Asp) residues of its substrates in the presence of broken DNA. Consistent with the accumulation of neuronal DNA damage, the brain samples of AD patients show increased levels of nuclear PARylation, suggesting a potential role of DNA damage-induced PARylation in neurons. Auto-PARylation dissociates PARP1 from the chromatin and inhibits its activity, while the sustained presence of PARP1 on the chromatin may trigger neurotoxicity. Because trans-RSV inhibited the auto-PARylation and depleted HPF1 along with serine-ADP-ribosylation, it was hypothesized that trans-RSVwould increase PARP1-dependent trans-PARylation of the chromatin. Trans-RSV increased the association of PARP1 with the chromatin and increased the levels of PARylated proteins in the chromatin fraction, which can be potentially considered as reminiscent of increased nuclear PARylation in AD brains. Further, low concentrations of trans-RSV (≤ 10 mM) and cis-RSV prevented the interaction of PARP1 with histone H3 while the higher concentrations (≥ 25 mM) of trans-RSV increased it. Although cis-RSV-mediated auto-PARylation of PARP1 resulted in its removal from the chromatin, unexpectedly, it was found that cis-RSV activated the de-ADP-ribosylation of the chromatin fraction along with higher levels of TyrRS. ADP-ribosyl-acceptor hydrolase 3 (ARH3) removes nuclear PARylation, and as expected, cis-and trans-RSV had differential effects on the recruitment of ARH3 to the chromatin. Despite having increased levels of nuclear PARylation, the levels of ARH3 remained unchanged in the hippocampal region of human AD patients, indicating that ARH3 may not be functional in human AD brain tissues in the absence of TyrRS. Consistently, it was found that TyrRS interacted with ARH3, suggesting a novel role of TyrRS in the removal of nuclear PARylation that enhances neuronal DNA repair and survival.

### ‘Trapped’ PARP1 Inhibits DNA Repair and Mediates the Neurotoxic Effects of Trans-RSV

Suicidal crosslinking of PARP1 to the damaged DNA causes neurotoxicity, and therefore, cell survival depends on removing ‘trapped’ PARP1 from the broken DNA by either ablation or auto-PARylation. Because trans-RSV caused DNA damage and inhibited the auto-PARylation of PARP1, and ablation of PARP1 rescues ‘trapped’ PARP1-mediated neurotoxicity, it was hypothesized that trans-RSV-mediated neurotoxicity is exerted through ‘trapped’ PARP1 on the damaged DNA. Consistently, small interfering RNA (siRNA) knockdown of PARP1 (siRNA^{PARP1}) protected against trans-RSV-mediated neurotoxicity but did not interfere with the effect of cis-RSV. These results indicate that while ‘trapped’ PARP1 mediates the neurotoxic effects of trans-RSV, cis-RSV effects are not dependent on PARP1 alone. Consistently, it was found that cis-RSVfacilitated the recruitment of PARP2 along with other DNA repair factors such as HPF1 and OGG1 to the chromatin while trans-RSV prevented their recruitment. However, PARP1 inhibits flap endonuclease (FEN1)-dependent long patch base excision repair (LP-BER), and consistently, cis-RSV decreased the recruitment of FEN1, indicating a potential role of TyrRS/ARH3-dependent de-ADP-ribosylation of PARP1 in limiting long patch BER. As further evidence of DNA repair, it was assessed whether DNA synthesis occurred following cis- or trans-RSVtreatment. The incorporation of nucleoside analogs into DNA from non-dividing neurons was previously used as a readout of neuronal DNA repair and depletion of PARP1 using siRNA or inhibition of PARP1 using small molecules increases the incorporation of nucleoside analogs. In agreement with the observation that ‘trapped’ PARP1 on the DNA impairs BER, trans-RSV prevented the incorporation of the nucleoside analog CldU into DNA fibers isolated from neurons, suggesting that DNA-repair associated synthesis is severely inhibited by trans-RSV. In contrast, CldU incorporation after cis-RSV treatment was detectable albeit reduced by 15-20% compared to control, suggesting the activation of PARP1-dependent short patch BER (SP-BER), potentially through the displacement of FEN1 and/or increased recruitment of unmodified PARP1 to the chromatin that limits DNA resection/repair.

### PARP Inhibitors Are Neurotoxic in HR-Deficient Post-Mitotic Neurons

Post-mitotic neurons are homologous recombination (HR)-deficient and utilize nonhomologous end-joining (NHEJ) for DNA repair. Interestingly, H3 serine-ADP-ribosylation facilitates NHEJ and Ku-dependent DNA repair is inhibited in AD brains. While PARP1 depletion increases HR, PARP inhibitors drive toxic NHEJ in HR-deficient cells in an ataxia-telangiectasia mutated (ATM)-dependent manner. Because it was previously shown that TyrRS activates ATM through acetylation and cis-and trans-RSV have opposite effects on TyrRS levels and the auto-PARylation of PARP1, the effect of well-known PARP inhibitors on cis-and trans-RSV-mediated effects on neurons was tested. While treatment with the PARP1-specific inhibitor AG-14361 (AG) did not affect cis-RSV-mediated neuroprotective effects, treatment with olaparib (Ola) (inhibits both PARP1 and 2) mitigated the neuroprotective effects of cis-RSV and did not affect trans-RSV-mediated neurotoxicity. Because siRNA^{PARP1} mitigated the effect of trans-RSV, taken together, these results indicate a critical neuroprotective role of PARP2, which may be utilized by cis-RSV in the absence of PARP1. Moreover, it was found that PARP inhibitors themselves decreased neuronal, induced neuronal DNA damage and neurite degeneration, suggesting that PARP inhibitors trigger cell death in post-mitotic HR-deficient neurons through the inhibition of H3 serine-ADP-ribosylation-dependent NHEJ.

Taken together, the mechanism for opposite effects of cis- and trans-RSV on neuronal survival emerging from our studies is illustrated in FIG. 8E. In this model, different forms of stress facilitate the interaction of TyrRS with PARP½ leading to their auto-serine-PARylation and subsequent removal from the damaged DNA allowing recruitment of DNA repair factors such as HPF1, ARH3, and OGG1 to repair the damage efficiently. cis-RSV-bound TyrRS facilitates the removal of auto-serine-PARylated PARP½ from chromatin while activating ARH3-mediated removal of ADP-ribose to achieve efficient neuronal DNA repair while limiting increased nucleotide incorporation/DNA repair and toxic NHEJ. In contrast, treatment with trans-RSV decreases TyrRS, in the absence of which PARP½ gets ‘trapped’ on the damaged DNA and impedes DNA repair leading to subsequent accumulation of DNA damage that drives neurodegeneration.

Synaptic protein synthesis is mainly regulated by monosomes. Therefore, strategies that increase the accumulation of neuronal monosomes may stimulate neurocognitive effects. Because cis-RSV induced the phosphorylation of eIF2α, the effect of cis-RSV on monosome formation in rat cortical neurons was tested. Interestingly, cis-RSV was found to stimulate the formation of monosomes, mimicking the effects of rapamycin which is known to induce monosome formation potentially by inhibiting global protein synthesis. Because Rab7a-associated late endosomes act as platform for mRNA translation in synapse-associated mitochondrial proteins, cis-RSV ability to increases the interaction of raptor with Rab7a was tested. Immunoprecipitation data showed that cis-RSV increases the association of raptor with Rab7a, providing a potential mechanism of monosome-associated translation of synaptic TyrRS.

Because the physiological levels of amyloid beta 42 (< 50 pM) is decreased in AD brains and Ab42 is essential for memory formation, the effect of physiological amyloid beta on neuronal TyrRS levels was tested. It was discovered that picomolar amyloid beta 42 (5-50 pM) is an endogenous stimulator of monosome formation, histone serine 10 ADP-ribosylation and neuronal TyrRS levels.

Taken together these data suggests that chromatin-bound TyrRS is an inhibitor of classic NHEJ and its removal by L-tyrosine facilitates neuronal DNA repair through classic NHEJ. Therefore, age-associated increase in serum tyrosine levels may increase classic NHEJ but may inhibit neuronal activity/Ab42-dependent SP-BER, which is essential for memory formation. Our study also implies that cis-RSV-mediated DNA repair may protect against DNA repair inhibitors that prevents PARP1-dependent DNA repair. For example, mammalian Rad52 promotes camptothecin (CPT)-induced cell death through the inhibition of PARP1-mediated single strand DNA break repair (SSBR). Rad52 strongly binds to single strand DNA (ssDNA) and activated PARP1 through its PAR chains and inhibits XRCC1-ligase III interaction. In the absence of Rad52, PARP-mediated SSBR protects against CPT-induced damage. Therefore, Rad52 interferes with SSBR, which promotes double strand DNA breaks (DSB) generation and neuronal cell death. However, Rad52 is essential for transcriptioncoupled homologous recombination (TC-HR) mediated through topoisomerase 1-mediated SSR, potentially facilitating short-patch base excision repair utilizing DNA polymerase beta and ligase III in a PARP1-dependent manner. Because most vertebrate/mammalian cells are not in S phase, they cannot depend on homologous recombination (HR)-dependent DNA repair processes. Therefore, they utilize PARP1/ligase III pathway to repair single strand DNA breaks. Therefore, PARP-mediated SSBR may play a major role in preventing DSB generation, thus promoting cell survival after CPT treatment/topoisomerase I-mediated transcriptional activation. Therefore, localization of PARP1 to TOPO I-generated single stranded DNA is essential to prevent unregulated recruitment of Ku and ligase IV (components of NHEJ) to the single ended DNA breaks and consistently, PARP1 inhibitors induce toxic NHEJ. Moreover, NHEJ can be a cytotoxic pathway in the presence of CPT. Therefore, PARP1 is essential to protect against topoisomerase I-mediated single strand DNA breaks while facilitating transcriptional activation. Consistently, BDNF protects against CPT-mediated neurotoxicity. These observations suggested that TyrRS/cis-RSV-mediated activation of PARP1 may facilitates the repair of SSB, induced by neurotoxic agents like Ab42 through the recruitment of ligase III to activate short-patch base excision repair (SP-BER). However, tyrosine/trans-resveratrol-bound form of TyrRS may facilitate the recruitment of ligase IV and Ku, which would exacerbate topoisomerase 1-mediated SSB and neurotoxicity.

### Discussion

This study shows that tyrosine is a negative regulator of TyrRS levels, thus providing a potential molecular basis for the decreased protein synthesis in AD brains, tyrosine-mediated cognitive impairments and inhibition of protein synthesis, increased oxidative DNA damage in aged neurons, and the circadian modulation of synaptic TyrRS. Interestingly, mutant amino acid transporter that accumulates tyrosine in Neurospora crassa is sensitive to tyrosine and its detoxification is essential for the survival of hematophagous insects. Because synaptic plasticity is regulated at the elongation step, it is conceivable that tyrosine-mediated regulation of TyrRS might be an evolutionary conserved negative feedback regulatory mechanism of protein synthesis exploited by neurons to enhance plasticity. Since tyrosine levels are decreased during the nadir/trough of circadian rhythm, these findings might also provide a molecular basis for sleep-stimulated brain protein synthesis and memory formation. Because CR lowers tyrosine levels, which is increased during aging, these results also provide a potential molecular basis for CR-mediated activation of BER and sleep-mediated activation of neuronal DNA repair.

Interestingly, exercise stimulates the production of BDNF, and DA is also known to activate PARP1-dependent DNA repair and stimulates protein synthesis in humans. Aging is the single most important contributing factor to the development of AD. Intriguingly, AD does not occur naturally in naked mole-rats (NMR), which are the longest-lived rodents resistant to AD. Because naked mole-rats maintain lower levels of tyrosine and higher levels of auto-PARylation of PARP1, it is tempting to speculate that decreased levels of tyrosine in naked mole-rats contribute to their longevity and resistance to AD through enhanced TyrRS/PARP1-dependent DNA repair, speculation that will need to be explored in the future. Although increased levels of branched-chain amino acids (BCAAs) are associated with metabolic disorders, in this context, it is interesting to note that the levels of BCAAs are decreased in AD as well as in ASD. Whether increased levels of tyrosine in children with ASD or mutations of amino acid transporter (LAT1) in the brain contribute to the increased incidence of mutations and dysregulated protein synthesis in ASD will be of future research interest. Since centenarians retain high levels of PARylation, which is also required for long-term memory formation, these observations suggest that decreased tyrosine levels may be an endogenous stimulator of TyrRS-mediated auto-PARylation of PARP1 and associated signaling events, which are dysregulated during aging and in neurocognitive and metabolic disorders. Therefore, tyrosine-mediated depletion of TyrRS and tyrosine-mediated induction of 8-oxo-dG, yH2AX and DNA damage shown here may have causal effects in human aging, motor, cognitive, and metabolic disorders.

However, this is the first time it has been demonstrated that D-tyrosine is a potent inhibitor of TyrRS/PARP1/HPF1-dependent serine-ADP-ribosylation and DNA repair, suggesting that D-tyrosine and its derivatives (including trans-RSV and its derivatives) would be ideal to inhibit PARP1-dependent DNA repair in cancer cells. This novel mechanism of action of D-tyrosine-mediated inhibition of HPF1-mediated serine-ADP-ribosylation might constitute the development of a novel class of PARP1 inhibitors that would inhibit different types of DNA repair pathways very efficiently.

This study also provides a potential molecular explanation for high dose trans-RSV-mediated brain volume loss in AD patients, worsening memory in schizophrenia, and increased CVD risk, similar to high concentrations of trans-RSV that depletes TyrRS and exacerbates neurotoxic effects. On the other hand, low-dose RSV studies that report beneficial cognitive benefits in postmenopausal women and protect against human heart failure used 50-75 mg twice a day dose of RSV (e.g., 6-9 µM RSV/dose) similar to low concentrations of trans-RSV that, like cis-RSV, increase TyrRS and provide neuroprotective effects in this study. Oxidative DNA damage is consistently elevated in CVD and PARP1-dependent DNA repair and is inhibited in mice models of heart failure. Because inflammation inhibits PARP1-dependent DNA repair, PARP1 modulates chromatin modification, and gene expression potentially regulates myelination, a CaMKII-dependent neurogenic program and long-term memory formation future studies are required to determine if these functions of PARP1 are affected in AD brains and contribute to the cis- and trans-RSV-mediated neuronal effects shown here.

Interestingly, cis-RSV induces the formation of monosomes similar to rapamycin. This data suggests that cis-RSV potentially increases neuronal TyrRS translation on Rab7a-coated late endosomes by increasing the association of raptor with Rab7a.

Picomolar Ab42 (5-50 pM) peptide was also found to be an endogenous stimulator of monosome formation, histone serine 10 ADP-ribosylation and neuronal TyrRS levels.

This study suggests that the PARP1/HPF1-dependent DNA repair pathway is decreased in the brains of AD patients, potentially providing a molecular basis for single strand DNA break-mediated neurodegeneration in AD brains and other neurodegenerative diseases. However, this study also indicates a potential role of Ab42 in the retention of topoisomerase I-mediated single strand DNA break through the recruitment of Rad52, which is essential for the transcription of long-neuronal genes, such as Ube3A, that are decreased in AD and in ASDs. This indicates a unique role of physiological Ab42 in sustaining topoisomerase I-mediated synaptic gene expression. Therefore, the inhibition of the Ab42 production using BACE inhibitors may stimulate neuronal DNA repair through classic NHEJ and PARP1-dependent alternative NHEJ, resulting in cognitive impairment by inhibiting the Topoisomerase I-mediated gene expression like Ube3A. Interestingly, histone serine 10 phosphorylation is also increased in cardiovascular diseases, suggesting that PARP1/HPF1-mediated histone serine ADP-ribosylation is inhibited during heart failure and other CVDs. Because PARP1 is essential to protect against topoisomerase I-mediated single strand DNA breaks and transcription-mediated DNA damage, and BDNF protects against CPT-mediated neurotoxicity and enhances cardio-protection after stroke, ischemia, reperfusion, and myocardial Infarction, agents that stimulate TyrRS-mediated activation of PARP1 and histone serine-ADP-ribosylation (like cis-resveratrol) may facilitate the repair of SSB induced by neurotoxic agents like Ab42 and tyrosine/phenylalanine through the recruitment of ligase III to activate SP-BER. However, strategies that increase the levels of Rad52 may prevent NHEJ and HR-mediated DNA repair and may possess anti-cancer effects without causing neurotoxicity. This might provide an avenue for the development of novel anti-cancer compounds without causing neuro/cardiotoxic effects. In contrast, tyrosine/trans-RSV-bound form of TyrRS may facilitate the recruitment of ligase IV and Ku, which would exacerbate topoisomerase 1-mediated SSB and cytotoxicity through toxic NHEJ, a mechanism exploited by currently available PARP1 inhibitors for anti-cancer treatments.

Strategies to inhibit tyrosine-mediated increase in neuronal DNA repair through NHEJ while stimulating TyrRS/PARP-dependent SP-BER during activity/Ab42/ topoisomerase I-mediated transcriptional activation may have therapeutic benefits in AD and other neurocognitive disorders driven by tyrosine/phenylalanine.

Therefore, in addition to a plausible explanation for the apparent benefits of low doses of trans-RSV and TyrRS being already nominated as a therapeutic target against AD by the National Institute on Aging’s Accelerating Medicines Partnership in Alzheimer’s Disease (AMP-AD) consortium (Agora, Sage Bionetworks), this study suggests that cis-RSV or compounds that use cis-RSV conformation as a pharmacophore may help in the chronotherapy of age-associated neurocognitive disorders and potentially degenerative and metabolic diseases of other tissues.

While certain embodiments of the disclosed subject matter have been described using specific terms, such description is for illustrative purposes only, and it is to be understood that changes and variations may be made without departing from the spirit or scope of the subject matter. In particular, this written description uses examples to disclose the presently disclosed subject matter, including the best mode, and also to enable any person skilled in the art to practice the presently disclosed subject matter, including making and using any devices or systems and performing any incorporated methods. The patentable scope of the presently disclosed subject matter is defined by the claims, and may include other examples that occur to those skilled in the art. Such other examples are intended to be within the scope of the claims if they include structural and/or step elements that do not differ from the literal language of the claims, or if they include equivalent structural and/or elements or steps with insubstantial differences from the literal language of the claims.

What is claimed:

1. A method of modulating a DNA damage response, comprising contacting a cell culture with a compound having Formula: 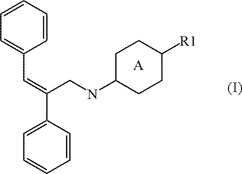
or a pharmaceutically acceptable salt and their isomers thereof, wherein:
ring A is a substituted or unsubstituted carbocyclyl, substituted or unsubstituted heterocyclyl, substituted or unsubstituted aryl, substituted or unsubstituted heteroaryl; and
R1 is 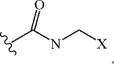 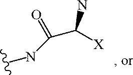 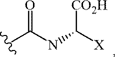
wherein X is an amino acid side chain.

2. The method of claim 1, the compound having the Formula: 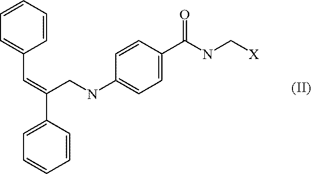
or a pharmaceutically acceptable salt and their isomers thereof.

3. The method of claim 1, the compound having the Formula: or 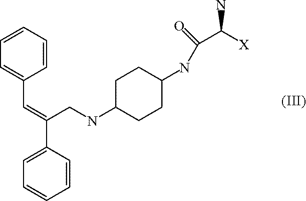
or a pharmaceutically acceptable salt thereof.

4. The method of claim 1, the compound having the Formula: 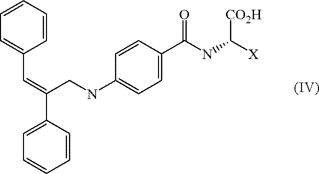
or a pharmaceutically acceptable salt and their isomers thereof.

5. The method of claim 1, wherein the amino acid side chain is selected from —H, —CH₃, —CH₂SeH, —CH₂CO₂H, —CH₂OH, —CH(CH₃)OH, —CH₂CH₂CO₂H, —CH₂CH₂CONH₂, 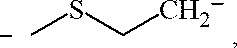 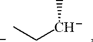 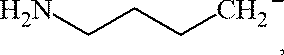 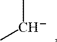 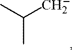 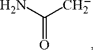 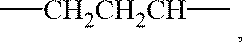 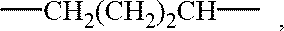 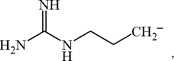 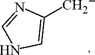 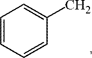 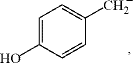 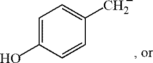 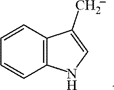
.

6. The method of claim 1, wherein the cell culture expresses poly-ADP-ribose polymerase 1 (PARP1).

7. The method of claim 1, wherein the compound is a resveratrol derivative.

8. A method of treating a disorder, the method comprising administering to a subject in need thereof a therapeutically effective amount of a pharmaceutical composition comprising a compound having Formula: 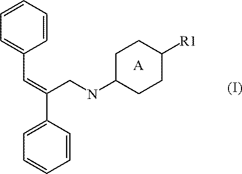
or a pharmaceutically acceptable salt and their isomers thereof, wherein:
ring A is a substituted or unsubstituted carbocyclyl, substituted or unsubstituted heterocyclyl, substituted or unsubstituted aryl, substituted or unsubstituted heteroaryl; and
R1 is 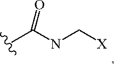 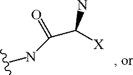 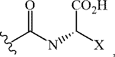
wherein X is an amino acid side chain.

9. The method of claim 8, the compound having the Formula: 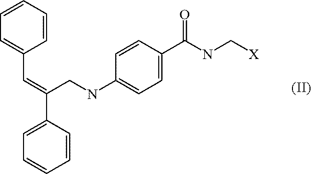
or a pharmaceutically acceptable salt and their isomers thereof.

10. The method of claim 8, the compound having the Formula: 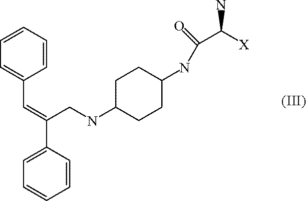
or a pharmaceutically acceptable salt thereof.

11. The method of claim 8, the compound having the Formula: 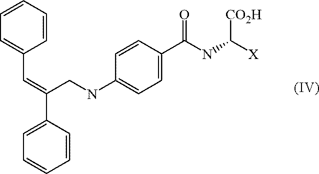
or a pharmaceutically acceptable salt and their isomers thereof.

12. The method of claim 8, wherein the amino acid side chain is selected from —H, —CH₃, —CH₂SeH, —CH₂CO₂H, —CH₂OH, —CH(CH₃)OH, —CH₂CH₂CO₂H, —CH₂CH₂CONH₂, 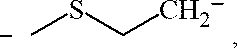 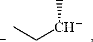 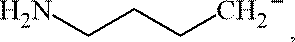 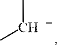 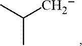 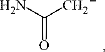 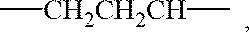 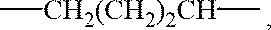 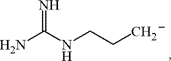 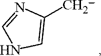 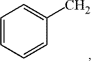 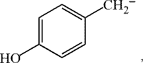 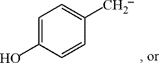 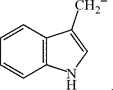
.

13. The method of claim 8, wherein the disorder is a neurocognitive and/or sleep disorder.

14. The method of claim 13, where in the neurocognitive and/or sleep disorder is selected from insomnia, hypersomnia, frontotemporal dementia, Alzheimer’s disease, Parkinson’s disease, amyotrophic lateral sclerosis, multiple sclerosis, Huntington’s disease, epilepsy and seizures, learning disabilities, neuromuscular disorders, Cockayne syndrome, cerebral palsy, dystonia, spinocerebellar ataxia with axonal neuropathy-1 (SCAN1), Angelman Syndrome, COVID-19-related neurocognitive problems, chemotherapy-associated neurocognitive problems including ‘chemo brain, autism spectrum disorder (ASD), delirium, mild-cognitive impairment, traumatic brain injury, phenylketonuria, or tyrosinemia.

15. The method of claim 8, wherein the disorder is a metabolic disorder.

16. The method of claim 15, wherein the metabolic disorder is selected from heart failure, cardiovascular disease, autoimmune-related disorders, myocardial ischemia reperfusion injury, hypertension, stroke, septic encephalopathy, diabetes, obesity, sepsis, Systemic Lupus Erythematosus or inflammation.

17. The method of claim 8, wherein the disorder is selected from breast cancer, ovarian cancer, colon cancer, pancreatic cancer, lung cancer, prostate cancer, brain tumor, leukemia, bone cancer and cachexia.

18. The method of claim 8, wherein the side chain is the side chain of an amino acid selected from a group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

19. The method of claim 8, wherein the compound is a D-tyrosine derivative.

20. The method of claim 8, wherein the compound is administered to the subject at a dose of from about 1 µM to about 100 µM.
